SEQUENCE LISTING (6 sequences)

SEQ ID NO: 1 - DNA, 22 bp, Artificial Sequence
  Primer SCF MF
acaccactgtttgtgctggatc

SEQ ID NO: 2 - DNA, 22 bp, Artificial Sequence
  SCF MR
aactgttaccagccaatgtacg

SEQ ID NO: 3 - DNA, 19 bp, Artificial Sequence
  GAPDH 5′primer
gtgaaggtcggagtcaacg

SEQ ID NO: 4 - DNA, 20 bp, Artificial Sequence
  GAPDH 3′ primer
gagatgatgacccttttggc

SEQ ID NO: 5 - DNA, 19 bp, Artificial Sequence
  PREP F primer
gctcgatacaataaacgcc

SEQ ID NO: 6 - DNA, 20 bp, Artificial Sequence
  EBV R primer
gtggtttgtccaaactcatc

(19) United States
(12) Patent Application Publication
(10) Pub. No.: US 2011/0020901 A1
(43) Pub. Date: Jan. 27, 2011

(54) Methods of Making Viral Particles Having a Modified Cell Binding Activity and Uses Thereof

(76) Inventor: Colin Maurice Casimir, London (GB)

Correspondence Address: NIKOLAI & MERSEREAU, P.A., 900 SECOND AVENUE SOUTH, SUITE 820, MINNEAPOLIS, MN, 55402, US

(21) Appl. No.: 12/821,686

(22) Filed: Jun. 23, 2010

Related U.S. Application Data
(63) Continuation of application No. 10/520,745, filed on Aug. 22, 2005, now abandoned.

(30) Foreign Application Priority Data
Jul. 11, 2002 (GB) 0216081.0

(51) Int. Cl. C12N 7/01

(52) U.S. Cl. 435/235.1

(57) ABSTRACT

The present invention relates to a method for packaging viral particles such that one or more peptides on the surface of the virus particle are derived from the packaging cell. By incorporating certain peptides it is possible to target viral particles to specific cell types. Such a system is of use, for example, in gene therapy treatments.

### CROSS-REFERENCE TO RELATED APPLICATION

This application is a continuation of application Ser. No. 10/520,745, filed 22 Aug. 2005, and prior PCT Application PCT/GB2003/003012, filed 11 Jul. 2003, entitled “METHODS OF MAKING VIRAL PARTICLES HAVING A MODIFIED CELL BINDING ACTIVITY AND USES THEREOF.

The present invention relates to a method for packaging viral particles such that one or more peptides on the surface of the virus particle are derived from the packaging cell. By incorporating certain peptides it is possible to target viral particles to specific cell types. Such a system is of use, for example, in gene therapy treatments.

The development of somatic gene therapy as a treatment for single gene inherited diseases and some acquired conditions, such as certain types of cancer, represents one of the most important technical advances in medicine.

Blood related disorders such as the X-linked immunodeficiencies, or chronic granulomatous disease (CGD), are amongst the most favourable candidates as model systems for the evolution of this technology. The general feasibility of gene therapy for disorders of this type has been amply demonstrated by the results obtained in the treatment adenosine deaminase dependent severe combined immunodeficiency (ADA-SCID) using peripheral blood T-cells.

However, many problems stand in the way of the realisation of the promise of these techniques. For example, in the experiments described above, the T-cells including the genes required by the patients are not immortal, requiring the therapy to be repeated at regular intervals. Further, attempts to effect a permanent correction, for example by gene transfer into pluripotent haematopoietic stem cells (PHSC), have thus far been unsuccessful.

Most of the clinical gene therapy trials that have been initiated to date have employed ex vivo strategies in which cells are genetically modified outside the body and reimplanted. The ability to deliver genes accurately and efficiently to selected target cell populations in vivo would greatly expand the scope of gene therapy, but current vectors are not well suited for this task.

Retroviral vectors offer a very high efficiency of chromosomal integration and are therefore well suited to gene therapy strategies, where it is a requirement that the therapeutic gene should be stably transmitted to future progeny of the target cell. Recombinant retroviruses have therefore been used in many clinical gene therapy protocols for ex vivo transduction of cultured T lymphocytes, fibroblasts, keratinocytes, hepatocytes, haemopoietic stem cells and neoplastic cells.

There are very few active clinical trials in which retroviral vectors or vector-producing cells are being used for in vivo transduction of neoplastic cells, by direct inoculation of tumour deposits or by instillation into a cavity (eg bladder, peritoneal or pleural space) whose wall is infiltrated by tumour. However, for in vivo transduction of lymphocytes, haematopoietic and other stem cells, vascular endothelial cells and disseminated malignancies, it will be necessary to develop retroviral vectors that adhere selectively to a target cell population when administered, especially when administered intravenously. Nontargeted vectors are inadequate since they will adhere predominantly to nontarget cells when introduced into the bloodstream leading to massive vector wastage and increasing the likelihood of undesirable side-effects.

Several approaches to developing retroviral vectors that can target specific cell types have received considerable attention in recent times.

It is possible to modify retroviral particles chemically to try and facilitate their entry into human target cells. Lactose coated β-galactosidase-transducing retroviral particles can specifically bind to the asialoglycoprotein receptor on the human HepG2 cells. However, the process is very inefficient and this form of chemical modification does not seem to offer a broad approach (Neda et al., 1991). Molecular bridges between the target cell and the transducing viral particles have also been tried to no effect (Goud et al., 1998) or at a cost of very low efficiency of transduction (Roux et al., 1989; Etienne-Juan et al., 1992).

An alternative approach is to engineer viral specificity by modifying the viral envelope glycoprotein binding site such that novel polypeptide sequences are displayed which confer a degree of target cell specificity. Such an approach has led to a widening of viral tropism, however, a disadvantage of modifying the viral envelope glycoprotein was a reduction in the efficiency of viral particle formation (titre) (Valsesia-Wittmann et al., 1994).

Alternatively, it is possible to replace the entire retroviral binding site with a new binding domain to confer an entirely new target cell binding activity to the retrovirus (Kasahara et al., 1994; Han et al., 1995; Matano et al., 1995). However, this approach has yet to be successfully repeated (Cosset and Russell, 1996).

Attempts have also been made to add new polypeptide sequences to the viral envelope by genetically modifying the viral genes encoding the surface glycoproteins. In this approach the native retroviral binding domain remains intact and novel target cell specific binding domains are added to the virus. Many polypeptide binding domains, including several single chain antibody fragments and polypeptide growth factors, have now been expressed as N-terminal extensions of the Moloney murine leukaemia virus (MLV) SU glycoproteins (Cosset et al., 1995; Schnierle et al., 1996; Somia et al., 1995; Russell et al., 1993; Marin et al., 1996; Valsesia-Wittmann et al., 1996). However, viral incorporation of such chimeras is usually reduced several-fold compared to wild-type envelopes, presumably reflecting a reduced efficiency of folding and/or oligomerisation (Cossett and Russell, 1996). Hence there appears to be a reduction in viral titre with this approach.

In a recent example of the latter approach, Gollan and Green (2002) attempted to modify MLV tropism by incorporating integrin receptor ligands into the viral envelope. Although the authors concluded that short ligands could be successfully introduced, such an approach is very arduous; in excess of 40 chimeric envelope derivatives were synthesised.

The present invention seeks to provide viral particles which exhibit a modified cell binding activity, that is, a cell binding activity which is different from that of the native virus binding activity.

In a first aspect, the present invention provides a method of making a viral particle having a modified cell binding activity comprising:
(i) providing a viral packaging cell containing viral nucleic acid encoding a viral particle having a first cell binding activity;
(ii) the viral packaging cell also containing nucleic acid encoding a passenger peptide binding moiety;
(iii) expressing the viral nucleic acid and nucleic acid encoding the passenger peptide binding moiety so that a viral particle buds from a packaging cell membrane and the passenger peptide binding moiety is provided at the cell membrane such that the passenger peptide binding moiety is incorporated into the viral particle to modify its first cell binding activity.

Surprisingly, simply expressing a desirable membrane bound peptide within a viral packaging cell allows one to effect incorporation of that peptide into viral particles which bud from a viral packaging cell membrane. Hence, the methods of the invention obviate the need to genetically engineer viral particles to express the peptide as a fusion with one or more proteins at the surface of a viral particle, eg the env protein of retroviruses. If the membrane bound polypeptide was, for example, a ligand that binds specifically to a target cell type, then the viral particle of the invention would specifically target that cell type. Accordingly, the viral particles of the invention also provide a means of delivering a bioactive agent to a target cell.

A further surprising benefit of the methods of the invention is that there appears to be no significant reduction in viral titre. As discussed above, existing methods of producing viral vectors with modified cell binding activity lead to a significant and undesirable reduction in viral titre.

Without wishing to be bound by any particular theory, we believe that the viral particles incorporate ‘passenger’ peptides into the viral envelope during viral budding.

The methods of the invention have the still further advantage that a single type of viral particle can have a common effect upon many different cell types in a cell type-specific manner: by simply transferring a single type of viral particle into different packaging cell lines that express a single target cell specific polypeptide on a cell surface membrane through which the virus particle buds, it is possible to manufacture a range of viral particles, each having a different cell type specific tropism conferred by the passenger peptide incorporated into its viral envelope. In other words, the invention allows one to select target cell specificity by selecting the viral packaging cell, rather than the existing approaches which involve genetically engineering a different viral particle.

It is preferred that the passenger peptide binding moiety is normally a membrane-bound peptide binding moiety. However, as described below, non-membrane peptide binding moieties can also be expressed as a fusion to polypeptides which incorporate a plasma membrane integration or ‘anchor’ region.

The packaging cell may be modified to introduce one or more passenger peptide binding moieties to be incorporated into the viral particle. For example, the packaging cell may express two or more different peptides having peptide binding moieties that can interact with the same target cell type, for example a human haematopoietic cell. In this way, it may be possible in increase the efficiency of targeting of the viral particle to a target cell type.

By ‘viral packaging cell’ we mean a cell in which a viral vector can be expressed and from which viral particles can be produced. Packaging cells may be any animal cells permissive for the virus, or cells modified so as to be permissive for the virus; or the packaging cell construct, for example, with the use of a transformation agent such as calcium phosphate. Cell lines which can be used as packaging cells for retroviruses included rodent cells such as NIH/3T3 cells and other murine cells; a suspension cell line such as Chinese Hamster Ovary cells (CHO) or L929 cells; and the FLY viral packaging cell system outlined in Cosset et al (1995) J Virol 69, 7430-7436.

For example, in the present invention a preferred retroviral packaging cell line is called Phoenix (Grignani et al., 1998; Kinsella and Nolan, 1996), discussed further in Example 2. The Phoenix ecotropic and amphotropic packaging cell lines used in the accompanying examples were kind gifts from Dr Gary Nolan, Stanford University, California, USA. These cell lines are second generation retrovirus-producer lines for the production of helper-free ecotropic and amphotropic retroviruses. The lines are based on the 293T cell line, a human embryonic kidney cell line.

A further preferred retroviral packaging cell line is the FLY viral packaging cell system outlined in Cosset et al (1995) J Virol 69, 7430-7436.

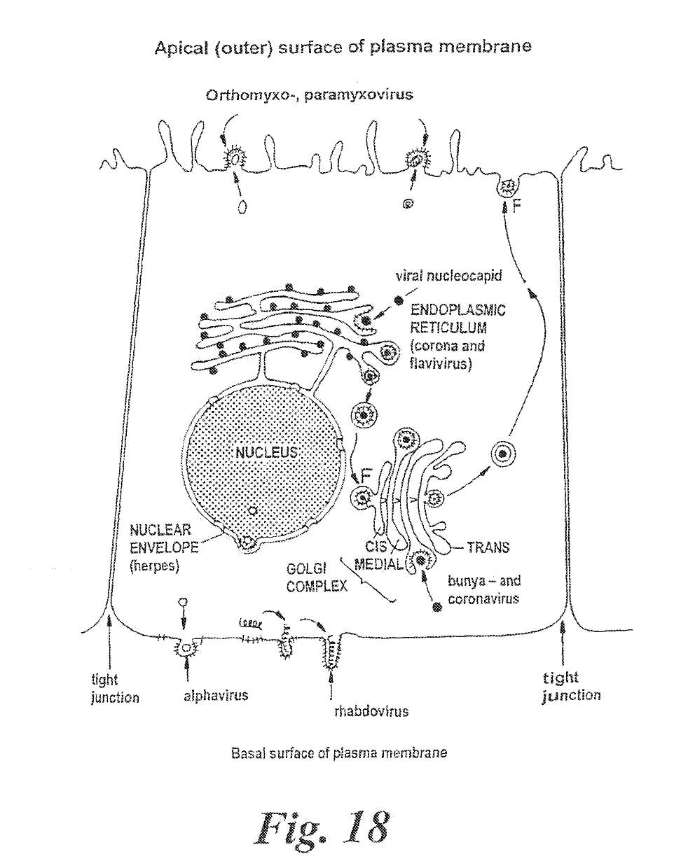

In a preferred embodiment the viral particle is derived from an enveloped-virus, for example retroviruses including rous sarcoma virus, human and bovine T-cell leukaemia virus (HTLV and BLV) and lentiviruses such as human and simian immunodeficiency viruses (HIV and SIV) and Mason-Pfizer monkey virus; foamy virus; herpes viruses (HSV, varicella-zoster, vaccinia); Pox viruses; orthomyxoviruses including influenza; paramyxoviruses including parainfluenza virus, respiratory syncytial virus, Sendai virus, mumps virus and measles virus; corona and flaviviruses; alphaviruses; rhabdoviruses including vesicular stomatitis virus and rabies virus; vaccinia viruses; bunyaviruses, and most RNA viruses, e.g. Rhabdovirus VSV (vesicular stomatitis virus). Also included are those enveloped viruses disclosed in FIG. 18 and the following documents: Dimmock and Primrose, (1987), Bodem et al., (1997), Strauss et al., (1995), Griffiths and Rottier (1992), Garoff et al., (1998) and Cadd et al., (1997).

By ‘first cell binding activity’ we mean the binding activity of the virus which dictates the natural host cell range of the virus. The host range of a virus generally is partially determined by a portion of the virus surface receptor moiety on the surface of the virion. Virus host range is further defined by the unique molecular biology of the infected cell by enhancers/promoters controlling gene expression. Some viruses attach to a specific cell type. The viruses produced by the packaging cell line will also attach to a specific cell type, by use of a natural surface receptor moiety.

By ‘binding moiety’ we mean a molecule that is available on the surface of the viral particle to bind to a molecule on a target cell. The ‘binding moiety’ may be a molecule on the virus or virus-like particle modified in such a way that its binding specificity is changed, or it may be a molecule added to, and exposed on the surface of, the viral particle to provide a new binding specificity.

By ‘passenger peptide binding moiety’ we mean a peptide with a binding moiety expressed by a viral packaging cell that is incorporated into the viral particle during viral budding from a cell membrane.

The term ‘peptide’ is also intended to embrace polypeptides and proteins.

By ‘modified cell binding activity’ we mean that the viral particle can interact with one or more different cell types than that of the unmodified viral particle. In this way, the cell binding activity (or ‘tropism’) of the viral particle may be modified such that the viral particle can bind to a wider range of cell types than the unmodified viral particle, or a narrower range of cell types.

Hence, the method of the invention can be applied to produce viral particles that have a modified cell binding activity such that the viruses can bind to a specific cell type.

By ‘packaging cell membrane’ we mean any of the membranes from which viral particles bud, including surface (outer) plasma membrane, nuclear envelope, endoplasmic reticulum, and/or golgi complex membrane. Although the majority of enveloped viruses acquire their envelope by budding from the outer plasma membrane, some, such as the herpesviruses, utilise the nuclear membrane listed here (see FIG. 18, adapted from Dimmock and Primrose, 1987).

In a preferred embodiment the packaging cell membrane to which the peptide binding moiety is targeted is the outer membrane.

By ‘membrane bound polypeptide’ we mean a polypeptide that is inserted into the membrane of a cell. Polypeptides can insert into a membrane most commonly because some of the amino acids that comprise the polypeptide are hydrophobic hence may stabily integrate into a lipid environment.

Non-membrane-bound polypeptides may be used in the invention if the polypeptides are fused to a region of polypeptides that have a membrane binding region. For example, the membrane-bound stem cell factor that is employed in one embodiment of the invention, and described in more detail in Example 1, may be used as a region of amino acids onto which a non-membrane bound polypeptide may be fused (an ‘anchor’). In this case, the hybrid polypeptide would be inserted into the plasma membrane of the cell and, hence, into the viral particles of the invention. Other membrane-bound growth factor trans-membrane regions may also be of use e.g. Flt-3 ligand, M-CSF (Lyman et al., 1995; Cosman et al., 1988)

Another polypeptide that can be used is the influenza haemaglutinin (Hatziioannou et al., 1999; Hatziioannou et al., 1998).

As was discussed above, the method of the invention can be used to modify the cell binding activity of a viral particle. By manipulating the cell binding activity it is possible that the viral particles produced by the method of the invention can bind to specific cell types. Therefore, one application of the method of the invention is to produce viral particles that can deliver one or more bioactive agents to specific cell types.

Hence in a preferred embodiment of the invention, the viral packaging cell line comprises additional nucleic acid which can be expressed to provide a bioactive agent which is active in or on a target cell.

By ‘bioactive agent’ we mean a molecule encoded by nucleic acid inserted into the viral genome which may have a biological effect upon a host cell or an organism, preferably a mammal, containing such a cell. Such biological effects include a therapeutic effect as well as other effects which may have utility for non-therapeutic applications. For example, the molecule could impart some therapeutic property upon any cell that the viral particle infects. In a further example, the desirable molecule could have a direct or indirect cytotoxic effect upon any cell that the viral particle infected. Examples of types of preferred bioactive agents are described below. Non-therapeutic effects may include, for example, biological functioning of the bioactive agent (eg β-galactosidase) to allow easy detection of a desired cell type. Such detection could be valuable for diagnostic applications as well as for experimental studies on target cells.

By ‘infects’ we mean that when the viral particle comes into contact with a target cell type its genetic material enters into that cell type. The ‘infection’ then proceeds according to the type of viral particle. For example, when a retroviral particle infects a target cell type, the retroviral genome is converted from RNA into DNA which is subsequently incorporated into the genome of the target cell. From this point the infection proceeds by the synthesis of new copies of the retroviral nucleic acid from the host genome, the retroviral nucleic acid is subsequently encapsulated with viral protein and bud from the outer plasma membrane of the target host cell.

Viruses that may be suitable for the methods of the invention are discussed in the following papers.

Garoff et al., (1998) discloses that alphaviruses, retroviruses, rhabdoviruses, orthomyxoviruses and paramyxoviruses bud from surface (outer) plasma membrane; coronaviruses bud from membranes between the endoplasmic reticulum and the golgi complex; while hepadnaviruses bud from the endoplasmic reticulum membrane. In particular, retroviruses, rhabdoviruses, orthomyxoviruses and paramyxoviruses are thought to incorporate host cell proteins during budding and may be particularly suitable in the methods of the invention. The disclosure of this document is incorporated herein by reference.

Bodem et al., (1997) describes Human Foamy Virus (HFV), the factors responsible for its preferential budding into cytoplasmic vesicles and its potential as a vector for genetic transfer. The disclosure of this document is incorporated herein by reference.

Griffiths and Rottier, (1992) reviews five groups, herpes-, rota-, corona-, bunya-, and pox-viruses that bud into, or assemble from, different compartments along the biosynthetic pathway. The review focuses on the virally-encoded membrane glycoproteins that are responsible for determining the site of virus assembly. The disclosure of this document is incorporated herein by reference.

By ‘nucleic acid’ we mean either DNA or RNA. If the viral particle of the invention is a DNA virus, we prefer that the nucleic acid is DNA. If the viral particle of the invention is an RNA virus, such as a retrovirus, then we prefer that the nucleic acid material is RNA.

Methods for inserting nucleic acid into the viral genome are well known in the art and are further described below (see, for example, Sambrook et al., MOLECULAR CLONING: A LABORATORY MANUAL. 2001. 3rd edition).

Although the viral particle of the invention can be derived from any enveloped virus, it is preferred that the viral particle is derived from a retrovirus. Such viruses are commonly used in gene therapies because their viral genome fully and stably integrates into the genome of any infected host cell. Hence any nucleic acids encoding bioactive agents would also be incorporated into the host cell genome.

Conveniently, the viral particle is Areplication-defective≅. By Areplication we mean a virus whose genetic material has been manipulated so that it cannot divide or proliferate in the cell it infects on its own. An advantage of such a viral particle is that the virus cannot multiply in the host cell or continue to infect other cells.

In a preferred embodiment the viral particle is derived from the retrovirus murine leukaemia virus (MLV).

Retroviral vectors encoding MLV are widely available to those skilled in the art, such as PINCO (Grignani et al., 1998) or the pBabe vector series (Morgenstern and Land, 1990). Alternatively, the present invention could employ a lentiviral vector such as those based on human immunodeficiency virus (HIV), caprine arthritis encephalitis virus (CAEV) or Visna-Maedi virus (VMV). Further vectors suitable for use in the methods described herein can be readily identified and/or prepared by the skilled person.

In another preferred embodiment the viral particle is derived from the human immunodeficiency virus (HIV). HIV vectors are well known and available to those skilled in the art.

The method of the invention can be employed to modify the cell binding activity of viral particles to deliver a bioactive agent to a target cell type. One such method of targeting a specific cell type is to incorporate a cell growth factor polypeptide on the viral particle. Hence a further embodiment of the invention is that one or more of the passenger peptide binding moieties is a cell growth factor.

By ‘cell growth factor’ we mean a peptide that acts to induce or repress the growth of a cell to which said peptide binds. Such a growth factor may be, for example, a ligand recognised or recognisable by a specific cell type. In such a case the ligand may act to promote proliferation of that cell type. Examples of such growth factors include, but are not limited to, platelet-derived growth factor (PDGF), fibroblast growth factor (FGF), interleukin 3 (IL-3), granulocyte-macrophage colony-stimulating factor GM-CSF, epidermal growth factor (EGF), FLT-ligand and stem cell growth factor (SCF), also known as mast cell growth factor, kit ligand factor, or Steel factor. Nucleic acid sequences encoding stem cell factors are described in WO92/00376, eg the −28 MGF stem cell factor.

A particularly preferred aspect of the invention is a method of modifying a viral particle so that it can bind to a specific stem cell type. In this way it may be possible to deliver a bioactive agent to a population of stem cells from which a wide range of other cells will differentiate.

Hence a particularly preferred growth factor is membrane-bound stem cell factor.

By ‘membrane bound stem cell factor’ we mean the peptide binding moiety discussed in Sehgal et al (1996). Nucleic acid sequences encoding stem cell factors are described in WO92/00376, eg the −28 MGF stem cell factor.

Stem cell factor (SCF) is a ligand that binds to the c-kit receptor protein found on the surface of quiescent stem cells (Hamel and Westphal, 1997). SCF exists in two forms, a longer soluble form and a shorter membrane bound form. The two forms results from differential mRNA splicing, with the soluble form having a proteolytic cleavage site coded by exon 6, while the membrane bound form has no exon 6 and thus no proteolytic cleavage site.

Thus, a preferred embodiment of the invention is a method of producing viral particle comprising a membrane bound stem cell factor within its viral envelope. Hence, the viral particle has a binding moiety that can interact with any cell type that has a molecule on the surface with which the membrane bound stem cell factor can bind. An example of such a molecule is the c-kit receptor. One example of a cell type with the c-kit receptor is pluripotent haematopoietic stem cells (PHSC), also known as haematopoietic stem cells (HSC).

Hence in a preferred embodiment the invention provides a method to target viral particles to haematopoietic stem cells. Such viral particles constitute a means ('vector') of delivering a bioactive agent to said cells.

Of course, using a peptide that acts as a growth factor as a means to modify the cell binding activity of a viral particle is only one such type of passenger peptide binding moiety. The method of the invention can also be used to modify the cell binding activity of a viral particle by adding an antibody, or an antigen binding fragment thereof, in which case the viral particles will take up the cell binding activity of the antibody or fragment thereof. Methods suitable for using antibody-derived cell binding moieties in viral particles are well known to those skilled in the art (Ager et al., 1996; Russell et al., 1993).

Hence a further preferred embodiment of the invention is a method of modifying the cell binding activity of a viral particle as described above wherein the peptide binding moiety is an antibody, or an antigen binding fragment thereof.

In one preferred embodiment the binding moiety has the binding activity of any one of a monoclonal antibody, ScFv (single chain Fv fragment), a dAb (single domain antibody) or a minimal recognition unit of an antibody.

The binding moiety may be a monoclonal antibody. Monoclonal antibodies which will bind to many of these antigens are already known but in any case, with today's techniques in relation to monoclonal antibody technology, antibodies can be prepared to most antigens. The binding moiety may be a part of an antibody (for example a Fab fragment) or a synthetic antibody fragment (for example, ScFv). Suitable monoclonal antibodies to selected antigens may be prepared by known techniques, for example those disclosed in AMonoclonal Antibodies: A manual of techniques≅; H Zola (CRC Press, 1988) and in AMonoclonal Hybridoma Antibodies: Techniques and Applications≅; J G R Hurrell (CRC Press, 1982).

Suitably prepared non-human antibodies can be Ahumanize≅ in known ways, for example by inserting the CDR regions of mouse antibodies into the framework of human antibodies.

The variable heavy (V_{H}) and variable light (V_{L}) domains of the antibody are involved in antigen recognition, a fact first recognised by early protease digestion experiments. Further confirmation was found by Ahumanization≅ of rodent antibodies. Variable domains of rodent origin may be fused to constant domains of human origin such that the resultant antibody retains the antigenic specificity of the rodent parental antibody (Morrison et al (1984) Proc. Natl. Acad. Sci. USA 81, 6851-6855). That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); ScFv molecules where the V_{H} and V_{L} partner domains are linked via a flexible oligopeptide (Bird et al (1988) Science 242, 423; Huston et al (1988) Proc. Natl. Acad. Sci. USA 85, 5879) and dAbs comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By AScFv molecules≅ we mean molecules wherein the V_{H} and V_{L} partner domains are linked via a flexible oligopeptide.

It may be advantageous to use antibody fragments, rather than whole antibodies. Effector functions of whole antibodies, such as complement binding, are removed. ScFv and dAb antibody fragments can be expressed as fusions with other polypeptides.

Minimal recognition units may be derived from the sequence of one or more of the complementary-determining regions (CDR) of the Fv fragment. Whole antibodies, and F(ab′)₂ fragments are Abivalent≅. By Abivalent≅ we mean that the said antibodies and F(ab′)₂ fragments have two antigen combining sites. In contrast, Fab, Fv, ScFv, dAb fragments and minimal recognition units are monovalent, having only one antigen combining sites.

From the above it can be seen that one application of the invention is a method of producing a viral particle having a cell binding activity conferred by an antibody. In this case, the viral particles produced may bind to any cell type to which the antibody or fragment thereof binds a cell surface antigen. Hence in a further embodiment of the invention, the peptide binding moiety of the viral particles recognises a target cell-specific surface antigen.

It will be appreciated by those skilled in the art that binding moieties which are polypeptides may be conveniently made using recombinant DNA techniques. The binding moiety may be fused to a protein expressed on a membrane of the packaging cell as disclosed above.

Nucleic acid sequences encoding many of the targeting moieties are known, for example those for peptide hormones, growth factors, cytokines and the like and may readily be found by reference to publicly accessible nucleotide sequence databases such as EMBL and GenBank. Once the nucleotide sequence is known it is routine for a person skilled in the art to make DNA encoding the chosen binding moiety using, for example, chemical DNA synthetic techniques or by using the polymerase chain reaction to amplify the required DNA from genomic DNA or from tissue-specific cDNA.

Many cDNAs encoding peptide hormones, growth factors, cytokines and the like, all of which may be useful as binding moieties, are generally available, for example from British Biotechnology Ltd, Oxford, UK.

Examples of cell surface antigens that may be bound by a viral particle produced using the method of the invention are shown in the table below. Also shown are the antibodies that are available that bind these antigens,

TABLE 1
Cell surface antigens for targeting

| | | |
|---|---|---|
| Antigen | Antibody | Existing uses |

a) Tumour Associated Antigens

| | | |
|---|---|---|
| Carcino-embryonic | C46 (Amersham) | Imaging and therapy of |
| Antigen | 85A12 (Unipath) | colon/rectum tumours. |
| Placental Alkaline | H17E2 (ICRF, Travers | Imaging and therapy of |
| Phosphatase | & Bodmer) | testicular and ovarian |
| | | cancers. |
| Pan Carcinoma | NR-LU-10 (NeoRx | Imaging and therapy of |
| | Corporation) | various carcinomas |
| | | including small cell |
| | | lung cancer. |
| Polymorphic Epithelial | HMFG1 (Taylor- | Imaging and therapy of |
| Mucin (Human milk | Papadimitriou, ICRF) | ovarian cancer and |
| fat globule) | | pleural effusions. |
| β-human Chorionic | W14 | Targeting of |
| Gonadotropin | | carboxypeptidase to |
| | | human xenograft |
| | | choriocarcinoma in |
| | | nude mice (Searle et al |
| | | (1981) Br. J. Cancer |
| | | 44, 137-144). |
| A carbohydrate on | L6 (IgG2a)¹ | Targeting of alkaline |
| Human Carcinomas | | phosphatase (Senter et |
| | | al (1988) PNAS USA |
| | | 85, 4842-4846. |
| CD20 Antigen on B | 1F5 (IgG2a)² | Targeting of alkaline |
| Lymphoma (normal | | phosphatase (Senter et |
| and neoplastic) | | al (1988) PNAS USA |
| | | 85, 4842-4846. |

b) Immune Cell Antigens

| | | |
|---|---|---|
| Pan T Lymphocyte | OKT-3 (Ortho) | As anti-rejection |
| Surface Antigen | | therapy for kidney |
| (CD3) | | transplants. |
| B-lymphocyte Surface | RFB4 (Janossy, Royal | Immunotoxin therapy |
| Antigen (CD22) | Free Hospital) | of B cell lymphoma. |
| Pan T lymphocyte | H65 (Bodmer and | Immunotoxin |
| Surface Antigen | Knowles, ICRF; | treatment of acute |
| (CD5) | licensed to Xoma | graft versus host |
| | Corp., USA) | disease, rheumatoid |
| | | arthritis. |

c) Infectious Agent-Related Antigens

| | | |
|---|---|---|
| Mumps virus-related | Anti-mumps | Antibody conjugated |
| | polyclonal antibody | to diphtheria toxin for |
| | | treatment of mumps. |
| Hepatitis B Surface | Anti HBs Ag | Immunotoxin against |
| Antigen | | hepatoma. |

¹Hellström et al (1986) Cancer Res. 46, 3917-3923
²Clarke et al (1985) Proc. Natl. Acad. Sci. USA 82, 1766-1770
Other antigens include alphafoetoprotein, Ca-125 and prostate specific antigen.

Since it is possible using the method of the invention to produce viral particles that can bind to specific cell types, one application of the invention is to employ the viral particles to introduce into the target cell types a bioactive agent that may be of benefit in the detection, prevention and/or treatment of a disorder associated with the target cell. Examples of cell types that mediate specific disorders are given in the table above.

Further examples of the types of diseases and disorders that may be treated by virus particles used according to the invention are given below.

A further aspect of the invention is a viral particle having a modified cell binding activity obtainable by the methods of the invention described above, the modified cell binding activity being conferred by a non-viral peptide other than a chimaeric viral polypeptide made by fusing the native env portion of the viral peptide with a peptide binding moiety. Such excluded viral particles include, inter alia, those disclosed in WO97/12049 (whose contents are herein incorporated by reference, amongst other things, for the purpose of possible amendment).

By ‘other than a chimaeric viral envelope polypeptide’ we mean that the modified cell bind activity of the viral particle is not provided by a polypeptide that has been fused to a viral envelope polypeptide encoded by the viral nucleic acid using conventional genetic engineering techniques. Rather, as described above the invention modifies the cell binding activity of a viral particle using a peptide binding moiety expressed by the viral packaging cells.

In a preferred embodiment the specific target cell types that the viral particles bind are mammalian cells, preferably human cells, more preferably quiescent cells. Hence the viral particles of the invention can bind to mammalian quiescent cells.

Advantageously, the specific target cell types that the viral particles bind are human haematopoietic stem cells.

In this application, “quiescent” refers to cells that are unlikely to enter mitosis within the next 24 hours in the absence of appropriate growth stimulus. Preferably, the population of quiescent cells is enriched in haematopoeitic stem cells, for instance by employing the isolation method of Beradi et al (1995) Science 267:104-108 using bone marrow cells. Other quiescent cell types suitable for use in the invention include resting T-lymphocytes, B-lymphocytes and monocytes, stem cells of non-haematopoietic tissues such as liver and muscle, epithelial stem cells in skin, gut, bladder and airways, vascular endothelial cells, quiescent neoplastic cells and germ cells such as sperm progenitors.

In one preferred embodiment, the viral particle includes nucleic acid which encodes a bioactive molecule having a directly or indirectly cytotoxic effect on a target cell. By Adirectly or indirectly≅cytotoxic, we mean that the molecule is itself toxic (for example ricin; tumour necrosis factor; interleukin-2; interferon-gamma; ribonuclease; deoxyribonuclease; Pseudomonas exotoxin A; caspase, etc), or it may be metabolised to form a toxic product, or it may act on something else to form a toxic product. The sequence of ricin cDNA is disclosed in Lamb et al (1985) Eur. J. Biochem. 148, 265-270 incorporated herein by reference.

For example, it would be desirable to target a DNA sequence encoding an enzyme using the virus or virus-like particle of the invention, the enzyme being one that converts a relatively non-toxic prodrug to a toxic drug. The enzyme cytosine deaminase converts 5-fluorocytosine (5FC) to 5-fluorouracil (5FU) (Mullen et al (1922) PNAS 89, 33); the herpes simplex enzyme thymidine kinase sensitises cells to treatment with the antiviral agent ganciclovir (GCV) or aciclovir (Moolten (1986) Cancer Res. 46, 5276; Ezzedine et al (1991) New Biol 3, 608). The cytosine deaminase of any organism, for example E. coli or Saccharomyces cerevisiae, may be used.

Thus, in a preferred embodiment of the invention, the gene encodes a cytosine deaminase and the patient is concomitantly given 5FC. By Aconcomitantly, we mean that the 5FC is administered at such a time, in relation to the transformation of the tumour cells, that 5FC is converted into 5FU in the target cells by the cytosine deaminase expressed from the said gene. A dosage of approximately 0.001 to 100.0 mg 5FC/kg body weight/day, preferably 0.1 to 10.0 mg/kg/day is suitable.

Components, such as 5FC, which are converted from a relatively non-toxic form into a cytotoxic form by the action of an enzyme are termed Apro-drugs≅.

Other examples of pro-drug/enzyme combinations include those disclosed by Bagshawe et al (WO 88/07378), namely various alkylating agents and the Pseudomonas spp. CPG2 enzyme, and those disclosed by Epenetos & Rowlinson-Busza (WO 91/11201), namely cyanogenic pro-drugs (for example amygdalin) and plant-derived β-glucosidases.

Enzymes that are useful in this embodiment of the invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as abzymes, can be used to convert the prodrugs of the invention into free active drugs (see, e.g. R J Massey, Nature, 328, pp. 457-458 (1987)).

Similarly, the prodrugs of this invention include, but are not limited to, the above-listed prodrugs, e.g., phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted by the enzyme of the conjugate into the more active, cytotoxic free drug. Examples of cytotoxic drugs that can be derivatized into a prodrug form for use in this invention include, but are not limited to, etoposide, teniposide, adriamycin, daunomycin, caminomycin, aminopterin, dactinomycin, mitomycins, cis-platinum and cis-platinum analogues, bleomycins, esperamicins (see U.S. Pat. No. 4,675,187), 5-fluorouracil, melphalan and other related nitrogen mustards.

In a further embodiment the gene delivered to the target cell encodes a ribozyme capable of cleaving targeted RNA or DNA. The targeted RNA or DNA to be cleaved may be RNA or DNA which is essential to the function of the cell and cleavage thereof results in cell death or the RNA or DNA to be cleaved may be RNA or DNA which encodes an undesirable protein, for example an oncogene product, and cleavage of this RNA or DNA may prevent the cell from becoming cancerous.

Ribozymes which may be encoded in the genomes of the viruses or virus-like particles herein disclosed are described in Cech and Herschlag ASite-specific cleavage of single stranded DNA≅ U.S. Pat. No. 5,180,818; Altman et al ACleavage of targeted RNA by RNAse≅ U.S. Pat. No. 5,168,053, Cantin et al ARibozyme cleavage of HIV-1 RNA≅ U.S. Pat. No. 5,149,796; Cech et al ARNA ribozyme restriction endoribonucleases and methods≅, U.S. Pat. No. 5,116,742; Been et al ARNA ribozyme polymerases, dephosphorylases, restriction endonucleases and methods, U.S. Pat. No. 5,093,246; and Been et al ARNA ribozyme polymerases, dephosphorylases, restriction endoribonucleases and methods; cleaves single-stranded RNA at specific site by transesterification≅, U.S. Pat. No. 4,987,071, all incorporated herein by reference.

In a still further embodiment the gene delivered to the target cell encodes an antisense RNA.

By Aantisense RNA≅ we mean an RNA molecule which hybridises to, and interferes with the expression from a mRNA molecule encoding a protein or to another RNA molecule within the cell such as pre-mRNA or tRNA or rRNA, or hybridises to, and interferes with the expression from a gene.

Conveniently, a gene expressing an antisense RNA may be constructed by inserting a coding sequence encoding a protein adjacent a promoter in the appropriate orientation such that the RNA complementary to mRNA. Suitably, the antisense RNA blocks expression of undesirable polypeptides such as oncogenes, for example ras, bcl, src or tumour suppressor genes such as p53 and Rb.

It will be appreciated that it may be sufficient to reduce expression of the undesirable polypeptide rather than abolish the expression.

It will be further appreciated that DNA sequences suitable for expressing as antisense RNA may be readily derived from publicly accessible databases such as GenBank and EMBL.

In another embodiment of the invention, the gene replaces the function of a defective gene in the target cell.

There are several thousand inherited genetic diseases of mammals, including humans, that are caused by defective genes. Examples of such genetic diseases include cystic fibrosis, where there is known to be a mutation in the CFTR gene; Duchenne muscular dystrophy, where there is known to be a mutation in the dystrophin gene; sickle cell disease, where there is known to be a mutation in the HbA gene. Many types of cancer are caused by defective genes, especially protooncogenes, and tumour-suppressor genes that have undergone mutation.

Thus, it is preferred that the viral particle of the invention, which may be useful in the treatment of cancer, contains a functional protooncogene, or tumour-suppressor gene to replace the function of the defective protooncogene or tumour-suppressor gene.

Examples of protooncogenes are ras, src, bcl and so on; examples of tumour-suppressor genes are p53 and Rb.

Typically, the desired protein or polypeptide will be one that a patient is unable to synthesise in his or her body or does not synthesise in the usual amount. An example of this is the use of gene therapy to treat adenosine deaminase dependent severe combined immunodeficiency (ADA-SCID). However, the concepts described herein are applicable to situations in which the nucleic acid encodes a protein or polypeptide that binds a substance that is overexpressed in a patient's body, e.g. causing some harmful physiological effect, or a protein or polypeptide that can bind to a polypeptide that is produced in a patient's body in an inactive form to activate it or in an active form to inactivate it. Preferably, the use of the present invention in these applications has the advantage that the therapy provided by transfecting the stem cells is long lasting or permanent, thereby helping to avoid the need for frequently repeated treatment.

Diseases that might be treated using the methods and materials described herein include all forms of chronic granulomatous disease (CGD), all forms of severe combined immunodeficiency (SCID), hyper gamma globulinaemia syndrome (hyper IgM), Wiskott-Aldrich Disease (WAS), thallassaemia, sickle-cell anaemia, other anaemias due to deficiencies of red blood cell proteins, neutrophil defects due to failure to synthesise granule components, e.g. myeloperoxidase deficiency, haemophilia and other clotting disorders such as complement deficiencies, lysomal storage disorders, such as Gaucher's disease, Hurler's disease, and mucopolysaccharidosis, leukocyte adhesion deficiency (LAD), bare lymphocyte syndrome, cancer and AIDS.

Other applications of the invention include the genetic modification of haematopoietic stem cells to repopulate the immune system with genetically modified T-lymphocytes that resist HIV, the genetic modification of haematopoeitic stem cells to repopulate bone marrow with haematopoietic progenitors that resist the myelosuppressive effects of cytotoxic chemotherapy, and the genetic modification of Tlymphocytes with chimeric T-cell receptors to target cytotoxic T-cells against tumours or virally infected cells.

In a further aspect, the present invention provides compositions comprising viral particles set out above, in combination with a suitable carrier. In this aspect, the present invention provides pharmaceutical compositions suitable for delivering nucleic acid encoding a bioactive agent to a population of stem cells in vitro, e.g. to prepare engineered stem cells for subsequent implant into a patient. Alternatively, the composition could be used in vivo, to directly deliver the nucleic acid to a patient's own stem cells. In this case, the composition preferably comprises a viral vector incorporating the nucleic acid encoding a bioactive agent and displaying a growth factor on its surface.

By Agene≅ we mean a nucleic acid coding sequence that may contain introns, or fragment thereof, or cDNA, or fragment thereof.

It is preferred that the passenger binding peptide moiety on the surface of the viral particle and the bioactive agent encoded by the nucleic acid contained with the viral particle are both polypeptides that may be produced by the techniques of genetic engineering.

The nucleotide sequence encoding the bioactive agent is preferably made by an alteration of the viral genome. Similarly, the passenger peptide binding moiety expressed in the packaging cell may be engineered using the methods outlined below.

The nucleotide sequence may be synthesised de novo using solid phase phosphoramidite chemistry, but it is more usual for the nucleotide sequence to be constructed from two parts, the first encoding the desirable molecule and the second the virus or virus-like particle. Similarly, the peptide binding moiety expressed in the packaging cell can be fused to a membrane-bound polypeptide region using the methods disclosed here. The two parts may be derived from their respective genes by restriction endonuclease digestion or by other methods known by those skilled in the art such as the polymerase chain reaction.

A variety of methods have been developed to operatively link two nucleotide sequences via complementary cohesive termini. For instance, synthetic linkers containing one or more restriction sites provide a method of joining the two DNA segment together. Each DNA segment, generated by endonuclease restriction digestion, is treated with bacteriophage T4 DNA polymerase of E. coli DNA polymerase I, enzymes that remove protruding, 3′-single-stranded termini with their 3′-5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and lifted to an expression vector that has been cleaved with an enzyme that produces termini compatible with those of the DNA segment.

Synthetic linkers containing a variety of restriction endonuclease sites are commercially available from a number of sources including International Biotechnologies Inc, New Haven, Conn., USA.

A desirable way to generate the DNA encoding the desirable molecule of the invention and the peptide binding moiety expressed in the packaging cell is to use the polymerase chain reaction as disclosed by Saiki et al (1988) Science 239, 487-491.

In this method each of the DNA molecules are enzymatically amplified using two specific oligonucleotide primers which themselves become incorporated into the amplified DNA. The said specific primers may contain restriction endonuclease recognition sites which may then be used to join the said two DNA molecules using T4 DNA ligase as disclosed.

The present invention also relates to a host cell transformed with a genetic (preferably DNA construct) construct of the present invention. The host cell can be either prokaryotic or eukaryotic. Bacterial cells are preferred prokaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343). Preferred eukaryotic host cells include yeast and mammalian cells, preferably vertebrate cells such as those from a mouse, rat, monkey or human fibroblastic cell line. Yeast host cells include YPH499, YPH500 and YPH501 which are generally available from Stratagene Cloning Systems, La Jolla, Calif. 92037, USA. Preferred mammalian host cells include Chinese hamster ovary (CHO) cells available from the ATCC as CCL61, NIH Swiss mouse embryo cells NIH/3T3 available from the ATCC as CRL 1658, and monkey kidney-derived COS-1 cells available from the ATCC as CRL 1650.

Transformation of appropriate cell hosts with a DNA construct of the present invention is accomplished by well known methods that typically depend on the type of vector used. With regard to transformation of prokaryotic host cells, see, for example, Cohen et al (1972) Proc. Natl. Acad. Sci. USA 69, 2110 and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

Electroporation is also useful for transforming cells and is well known in the art for transforming yeast cell, bacterial cells and vertebrate cells.

For example, many bacterial species may be transformed by the methods described in Luchansky et al (1988) Mol. Microbiol. 2, 637-646 incorporated herein by reference. The greatest number of transformants is consistently recovered following electroporation of the DNA-cell mixture suspended in 2.5×PEB using 6250V per cm at 25 μFD.

Methods for transformation of yeast by electroporation are disclosed in Becker & Guarente (1990) Methods Enzymol. 194, 182.

Successfully transformed cells, ie cells that contain a DNA construct of the present invention, can be identified by well known techniques. For example, cells resulting from the introduction of an expression construct of the present invention can be grown to produce the cytotoxic gene product as defined in the invention. Cells can be harvested and lysed and their DNA content examined for the presence of the DNA using a method such as that described by Southern (1975) J. Mol. Biol. 98, 503 or Berent et al (1985) Biotech. 3, 208. Alternatively, the presence of the protein in the supernatant can be detected using antibodies as described below.

In addition to directly assaying for the presence of recombinant DNA, successful transformation can be confirmed by well known immunological methods when the recombinant DNA is capable of directing the expression of the protein. For example, cells successfully transformed with an expression vector produce proteins displaying appropriate antigenicity. Samples of cells suspected of being transformed are harvested and assayed for the protein using suitable antibodies.

Thus, in addition to the transformed host cells themselves, the present invention also contemplates a culture of those cells, preferably a monoclonal (clonally homogeneous) culture, or a culture derived from a monoclonal culture, in a nutrient medium.

When the genetic construct is a plasmid DNA construct it can be purified. The DNA construct of the invention is purified from the host cell using well known methods.

For example, plasmid vector DNA can be prepared on a large scale from cleaved lysates by banding in a CsCl gradient according to the methods of Clewell & Helinski (1970) Biochemistry 9, 4428-4440 and Clewell (1972) J. Bacteria 110, 667-676. Plasmid DNA extracted in this way can be freed from CsCl by dialyse against sterile, pyrogen-free buffer through Visking tubing or by size-exclusion chromatography.

Alternatively, plasmid DNA may be purified from cleared lysates using ion-exchange chromatography, for example those supplied by Qiagen. Hydroxyapatite column chromatography may also be used.

The viral particles produced using the method of the present invention can be modified to bind to specific cell types, as described above. One application of this feature of the viral particles is in a method of enriching/purifying target cell types from a larger population of cells.

Hence in a further aspect of the invention, the viral particles of the invention may be used as a means by which an enriched population of a target cell type from a larger population of cells is prepared, wherein: (1) viral particles of the invention, modified to bind specifically to target cells, are exposed to a population of cells comprising the target cell type to permit binding to the viral particles; (2) viral particles bound to target cells are then separated from the population of cells; (3) optionally, the viral particles are subsequently removed from the target cells.

By ‘enriching’ we mean that the proportion of target cells present in a sample cells after the method outlined above has been employed is substantially greater than the proportion of target cells present in a original population of sample cells.

The viral particles of the invention may be engineered to comprise, for example, a ligand, or a growth factor, or an antibody that may bind with a target cell type. Such a viral particle can then be used as part of an enrichment method as outlined above, which resembles conventional immunoassays familiar to skilled persons

A further aspect of the invention is a method of enriching the titre of viral particles incorporating a passenger peptide binding moiety from a population of viral particles obtainable by a method as claimed in any preceding claim comprising:

i) providing a support to which the passenger peptide binding moiety binds; and,
ii) exposing the population of viral particles to the support; and, optionally,
iii) isolating the viral particles which bind to the support from the viral particles which do not bind to the support.

By ‘enriching the titre’ we include the meaning that the titre of the viral particles incorporating a passenger peptide binding moiety is enriched to at least 10⁹ ifu/ml, preferably 10¹⁰ ifu/ml, more preferably 10¹¹ ifu/ml (‘ifu’ means ‘infection forming units’ or ‘infectious units’). It is possible to use high titre viral packaging cell systems, for example the FLY system outlined in Cosset et al (1995) J Virol 69, 7430-7436, to increase the titre of the viral particles of the invention.

By ‘a population of viral particles’ we mean viral particles which have been produced according to the methods of the invention.

The viral particles of the invention may be enriched from a larger population of viral particles, some of which may not have incorporated a passenger peptide binding moiety, using this aspect of the invention. Using a system such as, for example, an immunoaffinity column, a technique well known to those skilled in the art, an antibody to the passenger peptide can bind to those viral particles that have incorporated a passenger peptide. Once bound, viral particles that have not incorporated a passenger peptide binding moiety can be separated from the bound viral particles. Bound viral particles are subsequently released from the column. In this example the immunoaffinity column functions as a support to which the passenger peptide binding moiety binds.

An alternative method of enriching the titre of viral particles of the invention, which is included in this aspect of the invention, is through the use of magnetic microbeads, for example the magnetic beads marketed Miltenyi Biotech (a catalogue and products may be ordered online at www.miltenyibiotec.com). Here, the viral particles of the invention are first bound by an antibody which recognises the passenger peptide. The antibody/viral particle complexes are then bound to magnetic microbeads: this can be via streptavidin/biotin binding between the magnetic microbeads and the antibody, or a Protein G conjugation of the antibody/viral particle complex by the magnetic microbeads. Once the magnetic microbeads/antibody/viral particle complex has been formed, the magnetic microbeads can be separated from the population of unbound viral particles and the viral particles of the invention recovered, as would be appreciated by a person skilled in the art. In this example the microbeads function as a support to which the passenger peptide binding moiety binds

An alternative method of enriching the titre of viral particles of the invention could be the use target cells containing a component, for example the SCF receptor (c-kit) which is bound by the membrane-bound stem cell factor (SCF), to which the passenger peptide binding moiety can bind as a means to enrich viral particles of the invention from a larger population of viral particles. In this example the target cells function as a support to which the passenger peptide binding moiety binds.

Other methods to enrich viral particles that have incorporated a passenger peptide binding moiety will be readily apparent to those skilled in the art. Further examples of supports to which the passenger peptide binding moiety binds will also be readily apparent to those skilled in the art.

A further aspect of the invention is a preparation of viral particles obtainable by any of the previous methods of the invention enriched for viral particles incorporating a passenger peptide binding moiety, the preparation having a titre of the viral particles of at least 10⁵ ifu/ml.

In order of increasing preference, the preparation of viral particles has a viral titre of at least 10⁵ ifu/ml, 10⁶ ifu/ml, 10⁷ ifu/ml, 10⁸ ifu/ml, 10⁹ ifu/ml, 10¹⁰ ifu/ml, 10¹¹ ifu/ml, or more.

In a further embodiment of this aspect of the invention the preparation of viral particles further comprises a pharmaceutically acceptable excipient and/or carrier. An example of a suitable excipient is an isotonic buffer such as phosphate buffered saline at pH 7.2, with other suitable excipients being well known to those skilled in the art

A preparation of this aspect of the invention may be prepared by, for example, the method of enriching the titre of viral particles incorporating a passenger peptide binding moiety from a population of viral particles as defined above.

Using the method of the invention it is possible to incorporate a passenger peptide binding moiety that is an antigenic peptide onto the surface of the viral particles of the invention. Hence, a further aspect of the invention provides viral particles of the invention for use as a vaccine.

The vaccine may be administered directly into the patient, into the affected organ or systemically, or applied ex vivo to cells derived from the patient or a human cell line which are subsequently administered to the patient, or used in vitro to select a subpopulation from immune cells derived from the patient, which are then re-administered to the patient.

If the viral particles of the invention are administered to cells in vitro, it may be useful for the cells to be transfected so as to co-express immune-stimulating cytokines, such as interleukin-2. The viral particles of the invention may be substantially pure, or combined with an immune-stimulating adjuvant such as Detox, or used in combination with immune-stimulatory cytokines. Suitably, any viral particles of the invention administered to the patient are sterile and pyrogen free.

The vaccine may be administered without adjuvant. The vaccine may also be administered with an adjuvant such as BCG or alum. Other suitable adjuvants include Aquila's QS21 stimulon (Aquila Biotech, Worcester, Mass., USA) which is derived from saponin, mycobacterial extracts and synthetic bacterial cell wall mimics, and proprietory adjuvants such as Ribi's Detox. Quil A, another saponin-derived adjuvant, may also be used (Superfos, Denmark). It is preferred if the vaccine is administered without adjuvant. Other adjuvants such as Freund's may also be useful.

Associated with the above application of viral particles of the invention is a use of the viral particles to present antigenic peptides to T-cells. T-cell antigen recognition requires antigen presenting cells (APCs) to present antigen fragments (peptides) on their cell surface in association with molecules of the major histocompatibility complex (MHC). T cells use their antigen specific T-cell receptors (TCRs) to recognise the antigen fragments presented by the APC. Such recognition acts as a trigger to the immune system to generate a range of responses to eradicate the antigen which has been recognised.

Using the method of the invention, antigenic peptides may be inserted into the envelopes of viral particles.

Therefore, a further aspect of the invention is the use of the viral particles of the invention to present antigenic peptides to mammalian T-cells.

A further aspect of the invention provides a method of delivery of the viral particle which contains a gene encoding a molecule having an indirectly cytotoxic function to a target cell.

Suitably, the indirectly cytotoxic function is an enzyme that converts a prodrug to a toxic drug. With such viral particle, once the viral particle has bound to the target cells, delivered its nucleic acid to the cells, and expressed the indirectly cytotoxic functions, which typically takes a day or so, the pro-drug is administered. The timing between administration of the viral particle and the pro-drug may be optimised in a non-inventive way.

The dosage of the pro-drug will be chosen by the physician according to the usual criteria. The dosage of the viral particle will similarly be chosen according to normal criteria, and in the case of tumour treatment, particularly with reference to the type, stage and location of tumour and the weight of the patient. The duration of treatment will depend in part upon the rapidity and extent of any immune reaction to the virus or virus-like particle.

Some of the viral particles either in themselves, or together with an appropriate pro-drug, are in principle suitable for the destruction of cells in any tumour or other defined class of cells selectively exhibiting a recognisable (surface) entity. Examples of types of cancer that may be treated using the viral particles are cancer of the breast, prostate, colon, rectum, ovary, testicle and brain. The viral particles of the invention are principally intended for human use but could be used for treating other mammals including dogs, cats, cattle, horses, pigs and sheep.

A further aspect of the invention is the use of a viral particle according to the invention in the manufacture of a medicament for the treatment and/or prevention of ovarian cancer.

The viral particles of the invention can target ovarian cancer cells by binding to cell surface antigens present on the cell surface. Once so bound the viral particles can act as a means of delivering a bioactive agent to a target cell. Examples of how such a targeting system can work and examples of bioactive agents are discussed above and further below.

A high proportion of cancer cells, including ovarian cancer cells, express the SCF receptor (c-kit) on the surface of the cell. Other cancer cells expressing the SCF receptor (c-kit) on the surface of the cell include breast, small-cell lung carcinoma, gastrointestinal stromal tumours (GIST), germ cell tumours (eg testicular), acute myeloid leukaemia (AML) and mastocytosis melanoma. Hence it is possible to target cancer cells, including ovarian cancer cells, by incorporating a membrane-bound stem cell factor (SCF) into the viral particle produced by the methods of the invention. The incorporation of a membrane-bound stem cell factor (SCF) into viral particles produced by the methods of the invention is discussed above and in the accompanying examples. Hence a further embodiment of the use of the viral particles in the prevention and/or treatment of cancer, including ovarian cancer, is wherein the virus particles incorporates a membrane-bound stem cell factor (SCF) as a passenger peptide binding moiety.

Recent research has shown that OPCML, an opioid binding protein/cell adhesion molecule-like gene, is frequently somatically inactivated in epithelial ovarian cancer (Sellar et al (2003) OPCML at 11q25 is epigenetically inactivated and has tumor-suppressor function in epithelial ovarian cancer. Nat. Genet. 34(3):337-43). The authors suggest that OPCML is a tumour-suppressor gene, and that loss of OPCML reduces intercellular adhesion and accelerates cell growth at the ovarian surface epithelium, thereby promoting the early steps of ovarian carcinogenesis.

As discussed above, in an embodiment of the invention the viral particle includes a gene which replaces the function of a defective gene in the target cell, for example a tumour-suppressor gene to replace the function of the defective tumour-suppressor gene. Hence in a further embodiment of the use of the viral particles in the prevention and/or treatment of cancer, including ovarian cancer, the viral particle includes a gene encoding a OPCML polypeptide. Examples of genes which encode a OPCML polypeptide include the polynucleotide sequence disclosed in GenBank Accession Number NM_{—}002545.

A further aspect of the invention is a pharmaceutical composition comprising the viral particles of the invention and a pharmaceutically acceptable carrier. The pharmaceutical formulation is prepared in a form suitable for administration to a patient and is sterile and pyrogen free.

Whilst it is possible for the viral particles to be administered alone, it is preferable to present it as a pharmaceutical formulation, together with one or more acceptable carriers. The carrier(s) must be “acceptable” in the sense of being compatible with the compound and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

A further embodiment of the invention is the use of a viral particle according to the invention in the manufacture of a medicament for the diagnosis and/or treatment and/or prevention of a disease or a disorder, by virtue of the bioactive agent encoded by the nucleic acid of the viral particle.

In this aspect, the medicament comprising the viral particles is preferably administered by implantation into a patient's bone marrow or is administered by infusion into a patient's blood. In order to allow the viral particles to target the bone marrow when administered by infusion, advantageously, receptors such as integrins can be incorporated on the surface of the viral particles. Alternatively or additionally, the immunogenicity of the viral particles can be reduced by incorporating an immunosuppressive factor such as FAS-ligand on the viral particles surface which can bind to activated T-cell FASreceptors, triggering the T-cells to die by apoptosis.

FAS-ligand expressing allogeneic cell implants have previously been shown to resist immune mediated rejection.

In a further aspect, the present invention provides the use of a viral particle according to the invention in the manufacture of a medicament for the treatment and/or prevention of arthritis.

The viral particles of the invention can target arthritic cells by binding to cell surface antigens present on the cell surface. Once so bound the viral particles can act as a means of delivering a bioactive agent to a target cell. Examples of how such a targeting system can work and examples of bioactive agents are discussed above.

As outlined above, an example of a cell surface antigen found on arthritic cells that may be bound by a viral particle produced by the methods of the invention is the Pan T lymphocyte Surface Antigen (CD5). The cell surface antigen may be bound by incorporating an binding molecule which binds to CD5 into the viral particle produced by the methods of the invention, for example a H65 antibody. Hence an embodiment of this aspect of the invention is wherein the virus particles incorporates a binding molecule which binds to CD5 as a passenger peptide binding moiety.

Alternatively, other binding molecules, for example other antibodies, which bind CD5 can be produced using routine methods, as will be appreciated by a person skilled in the art, and can be incorporated into viral particles produced by the methods of the invention. The incorporation of antibodies into viral particles produced by the methods of the invention is discussed above.

In addition to this, it is possible to target non-arthritic cells which can act as a link between the lymphocytes that attack the joint and the cells of the joint itself. An example of such a non-arthritic cell type is mast cells, as discussed in Lee D M, Friend D S, Gurish M F, Benoist C, Mathis D, Brenner M B. Mast cells: a cellular link between autoantibodies and inflammatory arthritis. Science. 2002 Sep. 6; 297(5587):1689-92. The paper suggests that if the population of activated mast cells is eradicated then the inflammatory response and the T cells that attack the joint also subside.

SCF is a major regulator of the growth of mast cells which are, therefore, abundant expressers of the SCF receptor (c-kit). Hence it is possible to target mast cells by incorporating a membrane-bound stem cell factor (SCF) into the viral particle produced by the methods of the invention. The incorporation of a membrane-bound stem cell factor (SCF) into viral particles produced by the methods of the invention is discussed above and in the accompanying examples. Hence a further embodiment of this aspect of the invention is wherein the virus particles incorporates a membrane-bound stem cell factor (SCF) as a passenger peptide binding moiety.

In a further aspect, the present invention provides a method of transforming a population of quiescent cells with nucleic acid encoding a bioactive polypeptide so that the nucleic acid is incorporated into the genome of the cells, the method comprising exposing the cells to viral particles as described above, wherein the surface bound growth factor induces the cells to divide, so that the nucleic acid encoding the bioactive polypeptide for treating, preventing and/or diagnosis of a disease or disorder is incorporated into the genome of the cells.

In this aspect, preferably the quiescent cells are a population of bone marrow cells, optionally enriched for haematopoeitic stem cells.

In a further aspect, the present invention provides the use of a viral particle according to the invention in the manufacture of a medicament for diagnosing and/or treating a mammal having a defective gene, wherein nucleic acid encoding a molecule for treating a disease or disorder is inserted into the genome of a population of cells in vivo by implantation of a viral particle according to the invention into bone marrow or by infusion into a blood.

In a further aspect, the viral particles of the invention can be used for gene transfer. By ‘gene transfer’ we mean that the viral particles of the invention can be used to transfer nucleic acid encoding a bioactive agent to the genome of a target cell.

Embodiments of the invention will now be described in more detail by reference to the following non-limiting Figures and Examples, in which:

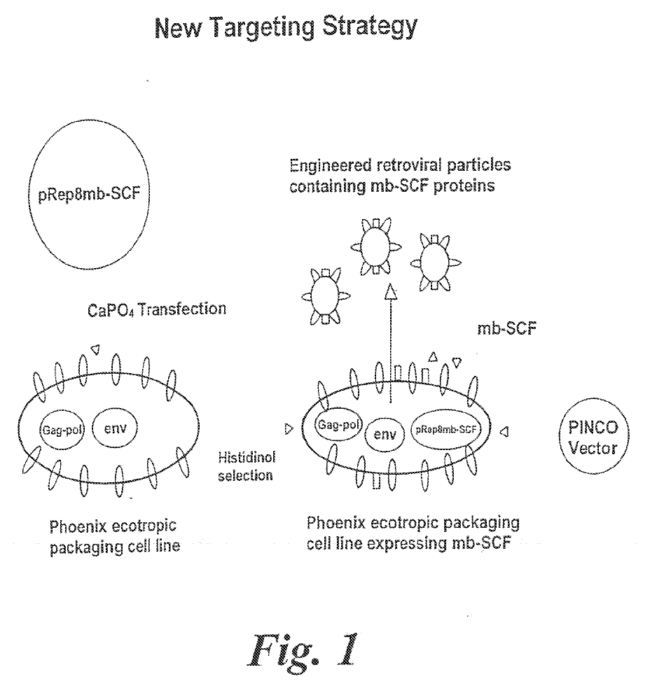

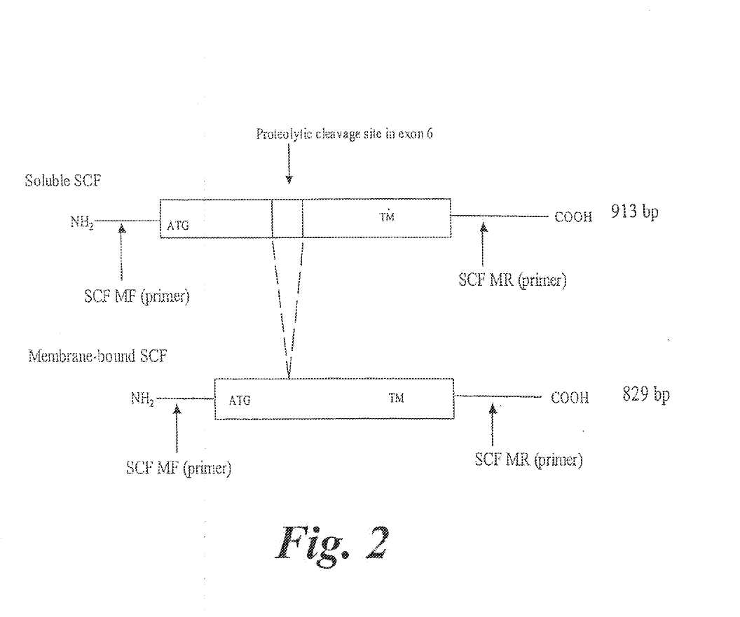

FIG. 1 Strategy for expression of biologically active growth factor on retroviral particle surface. The vectors shown and the methods employed are described further in the description.

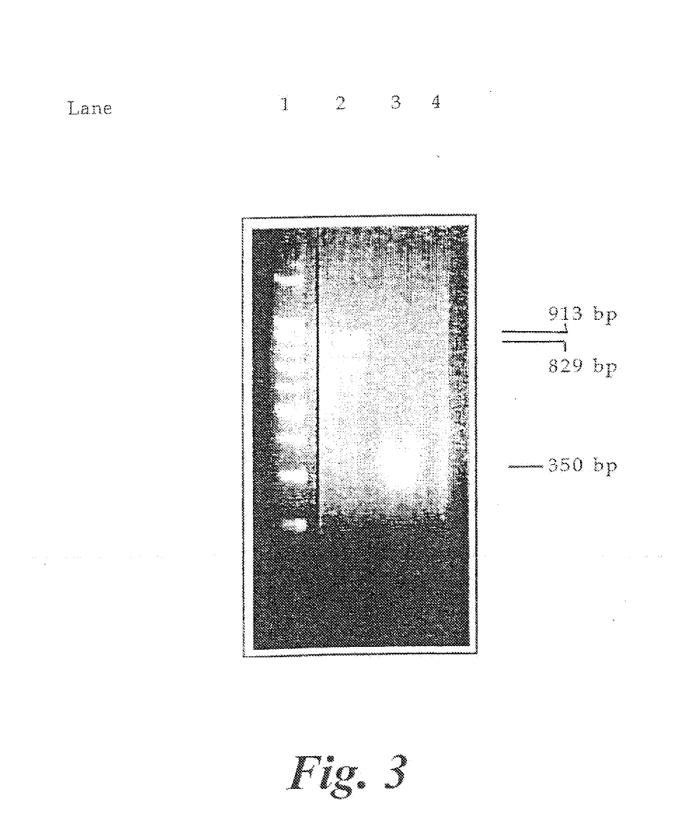

FIG. 2 Schematic diagram of stem cell factor (SCF) cDNA. The positions of the forward (SCF MF) and reverse primer (SCF MR) used to amplify the SCF cDNA are shown. Primer sequences are described in the description.

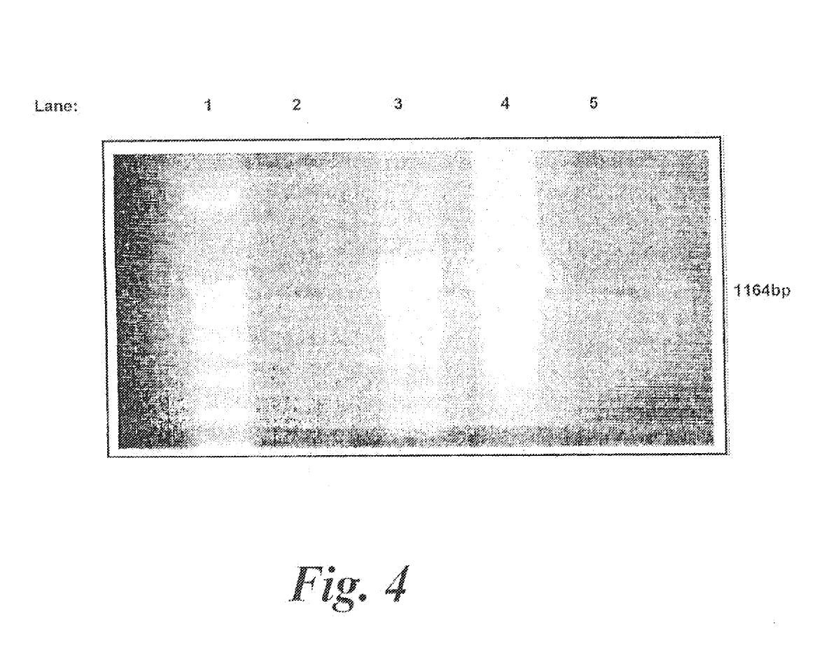

FIG. 3 Agarose gel electrophoresis of RT-PCR product from L88.5 RNA, amplified with SCF primers. L88.5 is a human cell line expressing soluble and membrane-bound forms of the stem cell factor (SCF). Lane 1-100 bp molecular weight marker; Lane 2-RT-PCR analysis of RNA extracted from L88.5 cells using SCF MF and MR primers. Three distinct bands were observed, a 913 bp fragment indicative of full length SCF encoding the soluble isoform; an 829 bp fragment encoding membrane-bound SCF (mb-SCF); and an unidentified fragment between 700 bp and 800 bp, a PCR artefact or possibly another splice variant of the SCF. The 829 bp (mb-SCF) fragment was cloned into a PCR cloning vector; Lane 3—Control PCR reaction from cDNA of L88.5, amplifying the GAPDH gene product of 350 bp using GAPDH specific primers; Lane 4-RT-PCR products from reverse transcribed cDNA without the reverse transcriptase as template.

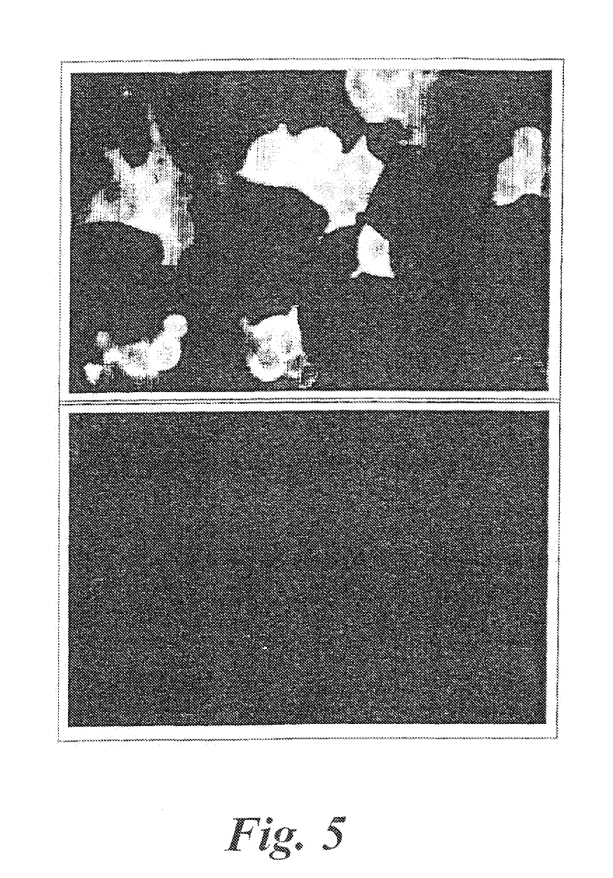

FIG. 4 PCR detection of recombinant plasmids in engineered packaging cells. The recombinant plasmid, pRep8 mb-SCF, was transfected by calcium phosphate precipitation into the Phoenix ecotropic cell line. Stable clones obtained were propogated and trypsined when cells were about 90% confluent. Two microlitres of a cell suspension (from clone 3) containing these cells were subjected to PCR analysis using PrepF and EBVR primers to detect the presence of the plasmids. Lane 1 —100 bp molecular weight marker; Lane 2—untransfected ecotropic packaging cells; Lane 3—Two microlitres of Phoenix ecotropic cell suspension, transfected with pRep8 mb-SCF; Lane 4 pRep8 mb-SCF control DNA as template for the PCR; Lane 5—negative control, ie water as template DNA. The expected amplified product of 1164 bp was observed in lanes 3 and 4.

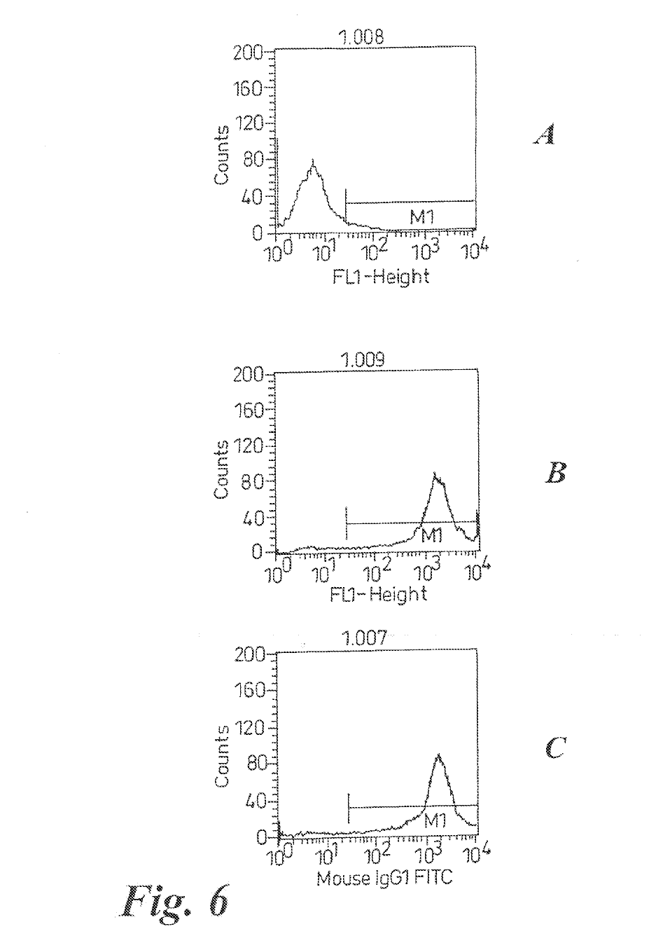

FIG. 5 Immunofluorescence assay of Phoenix ecotropic packaging cell line transfected with pRepmb-SCF. Top panel—transfected cells were grown on glass cover slips and fixed with methanol. The cover slips were stained with anti-SCF antibody, washed twice with PBS and stained with a fluorescent labelled secondary antibody. Cover slips were mounted onto glass slides and viewed under a UV microscope (mag 400×). The figure demonstrates intense surface staining. Bottom panel—negative controls of untransfected cells. These cells were not fluorescent. The exposure time required to capture the negative images (cells) had to be significantly increased.

FIG. 6 Flow cytometry analysis of retroviral packaging cells. Panel A—Unmodified ecotropic retroviral packaging cells. Panel B—Clone 3 packaging cells expressing high levels of mb-SCF proteins. Panel C—retroviral packaging cells expressing CD59.

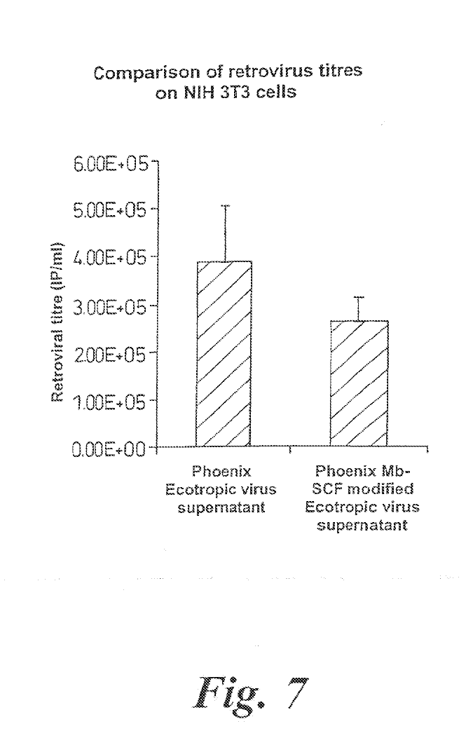

FIG. 7 Comparison of retroviral titres. Retroviral titres from 3 experiments were determined on NIH 3T3 cells. The mb-SCF modified packaging cells showed a slight but statistically insignificant decrease in titre (paired t test, p=0.5).

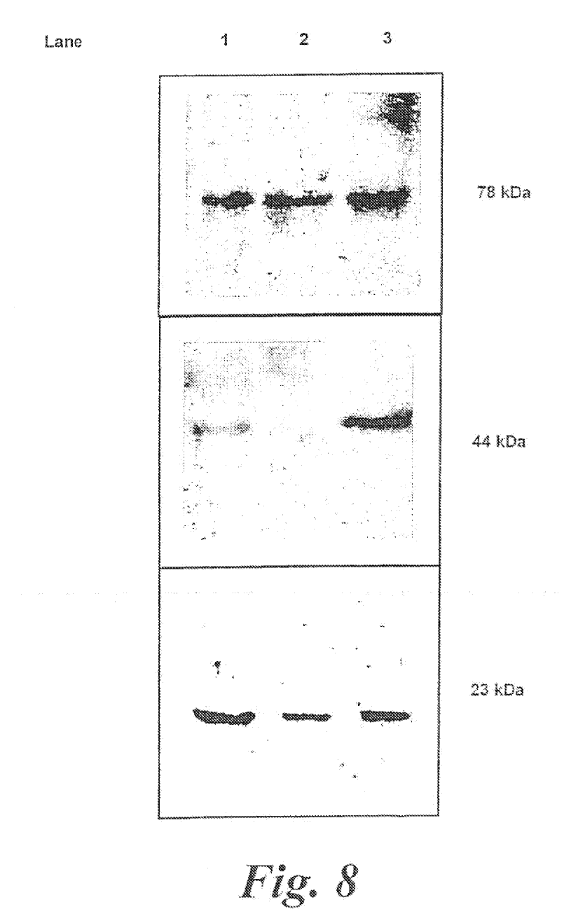

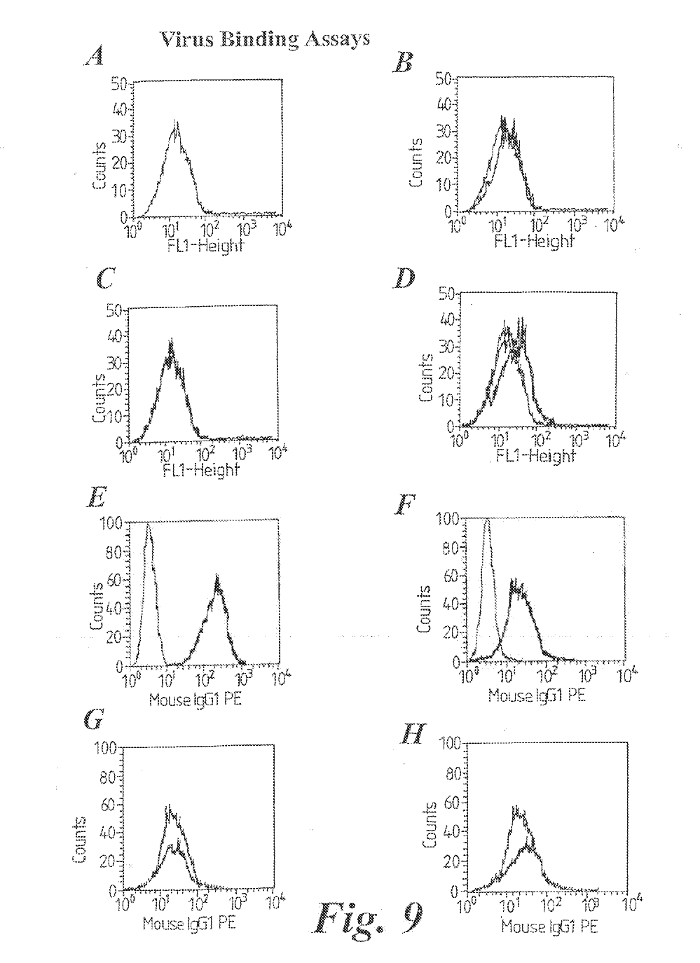

FIG. 8 Western blot analysis of retroviral particles. Top panel—retroviral particles expressing the SU portion of the envelope protein. Middle panel—retroviral particles expressing mb-SCF. Bottom panel—retroviral particles expressing CD59, a naturally occurring protein on the packaging cell. Lane assignments; Lane 1—retroviral particles obtained from stable mb-SCF producing ecotropic cells; Lane 2—unmodified ecotropic retroviral particles; Lane 3—retroviral particles obtained transiently from mb-SCF ecotropic packaging cells.

FIG. 9 Retroviral binding assays. Retrovirus was incubated with target cells and the cells stained for bound virus using anti-retroviral envelope monoclonal antibody. Panel B-D show the virus binding data (green trace) superimposed on the control spectrum (grey) from Panel A. Panels G and H show the virus binding data as the heavier black trace superimposed on the control (grey). A) Mo7e cells alone; B) Mo7e+Amphotropic Virus; C) Mo7e+Ecotropic Virus; D) Mo7e+SCF−Ecotropic Virus; Panels E and F. Staining of Mo7e cells with antibody to c-kit (black trace) compared to an isotype control (grey trace) before (E) and (F) overnight incubation with 100 ng/ml SCF. G) Mo7e cells from panel F+Ecotropic virus; H) Mo7e cells from panel F+Mo7e+SCF−Ecotropic Virus

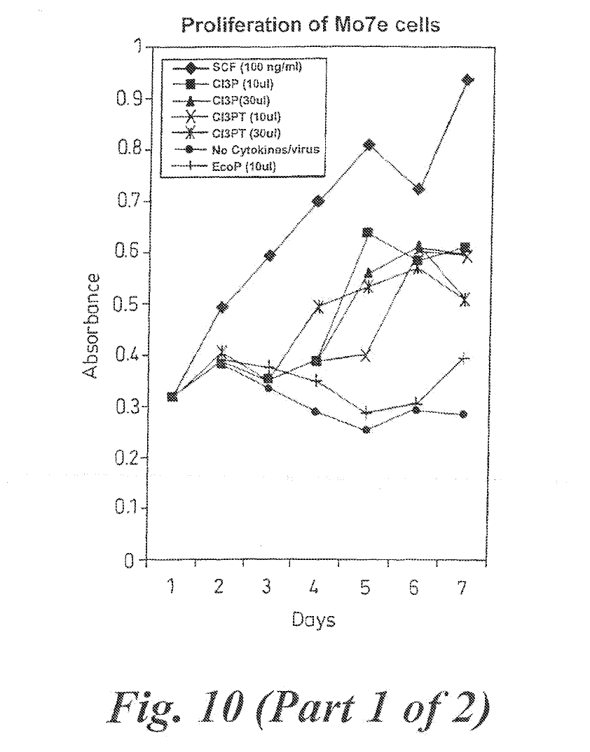

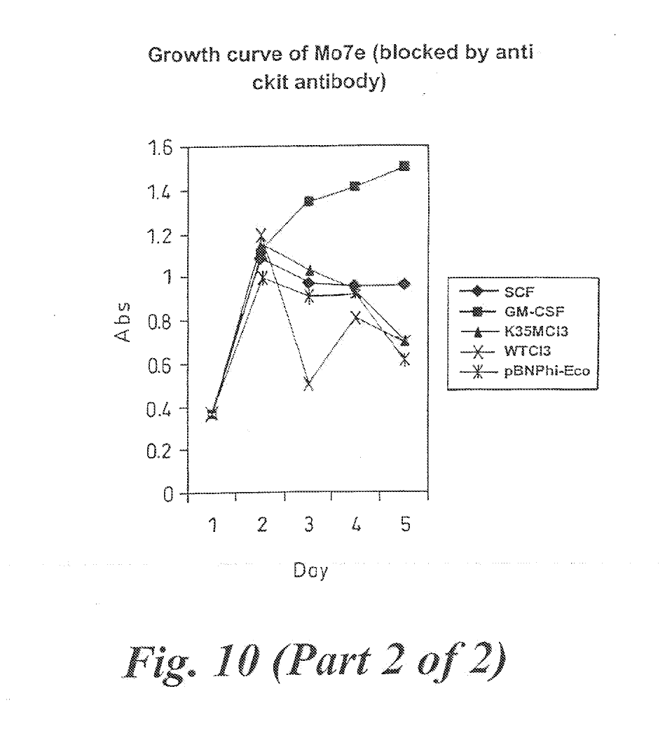

FIG. 10 Proliferation assays using retroviral particles incorporating surface SCF.

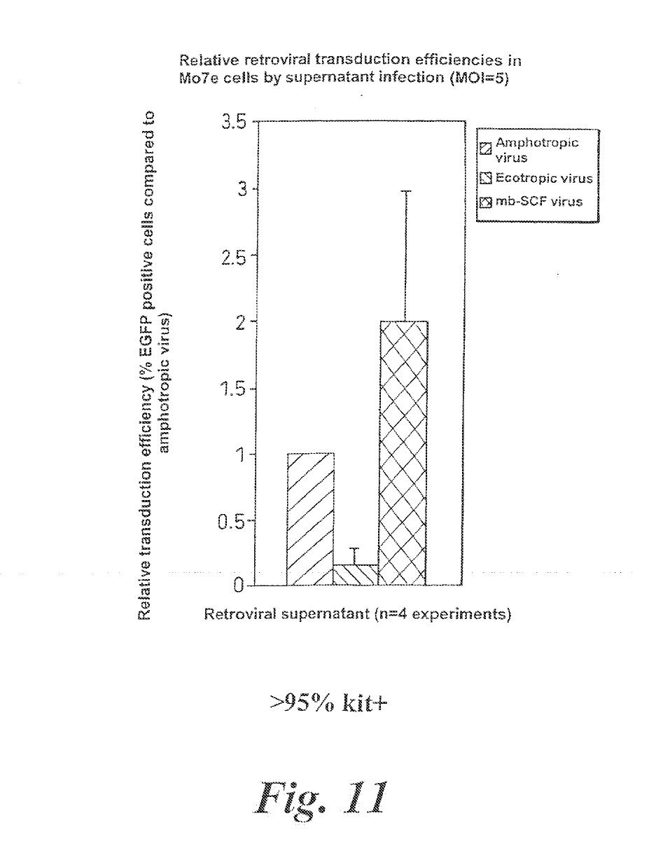

FIG. 11 Transduction efficiency of SCF—modified ecotropic retrovirus on human kit positive Mo7e cells.

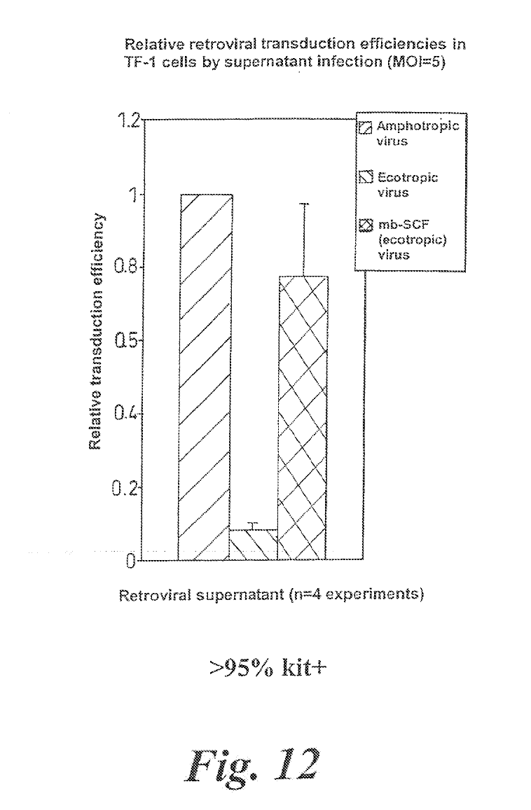

FIG. 12 Transduction efficiency of SCF—modified ecotropic retrovirus on human kit positive TF-1 cells.

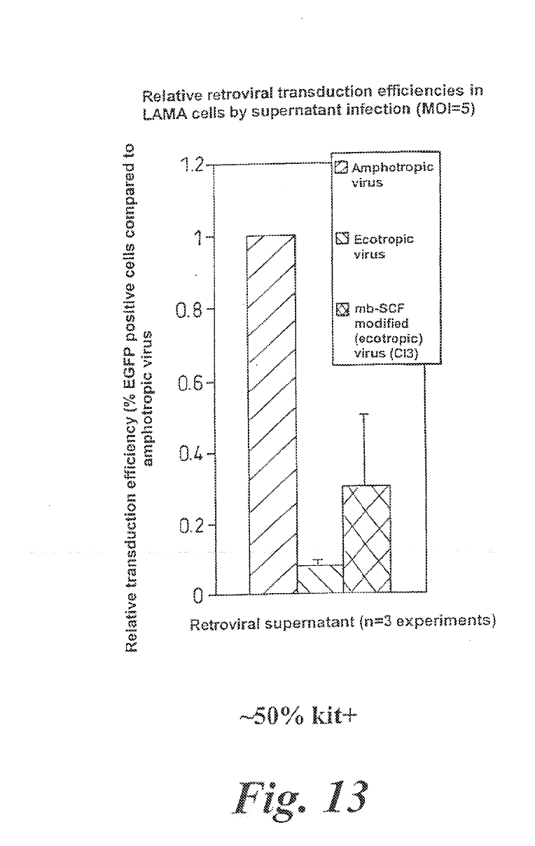

FIG. 13 Transduction efficiency of SCF—modified ecotropic retrovirus on human kit positive LAMA cells.

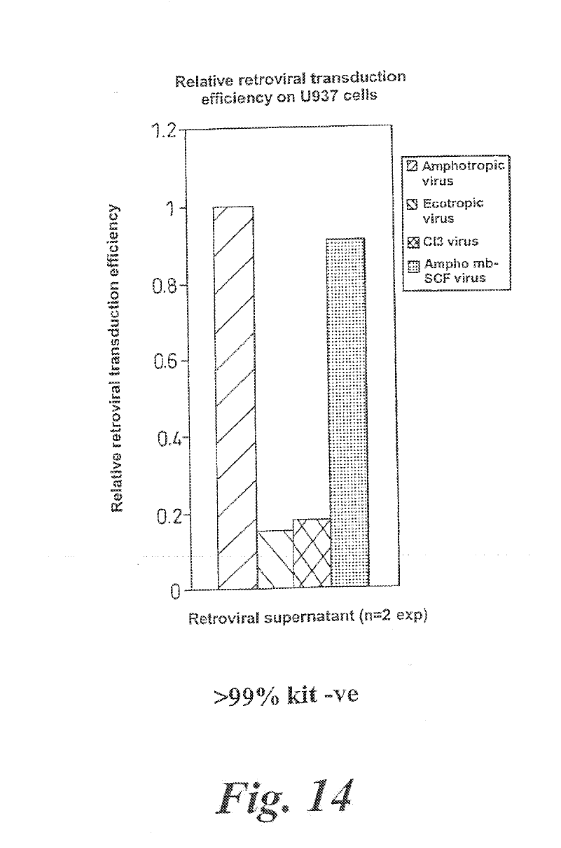

FIG. 14 Transduction efficiency of SCF—modified ecotropic retrovirus on human kit negative U937 cells.

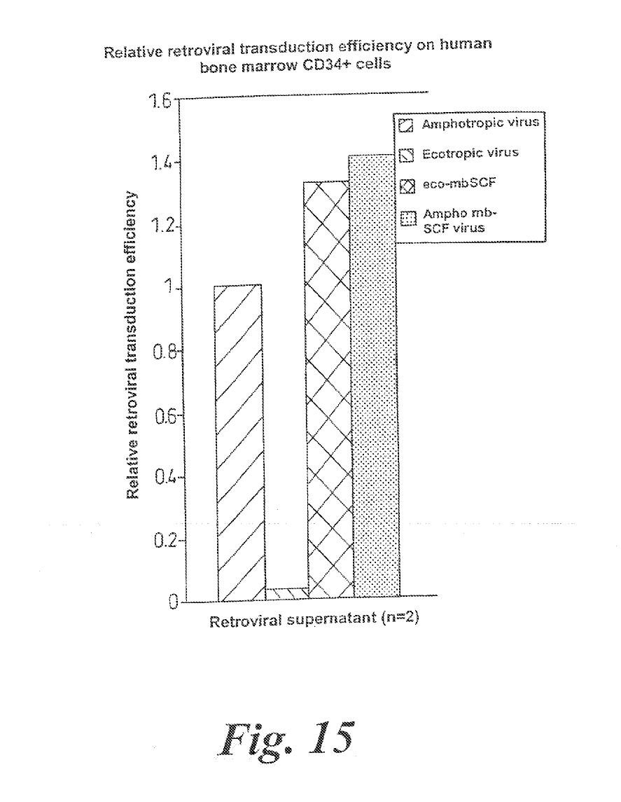

FIG. 15 Relative transduction efficiency of SCF—modified ecotropic virus on primary human haematopoietic progenitor cells.

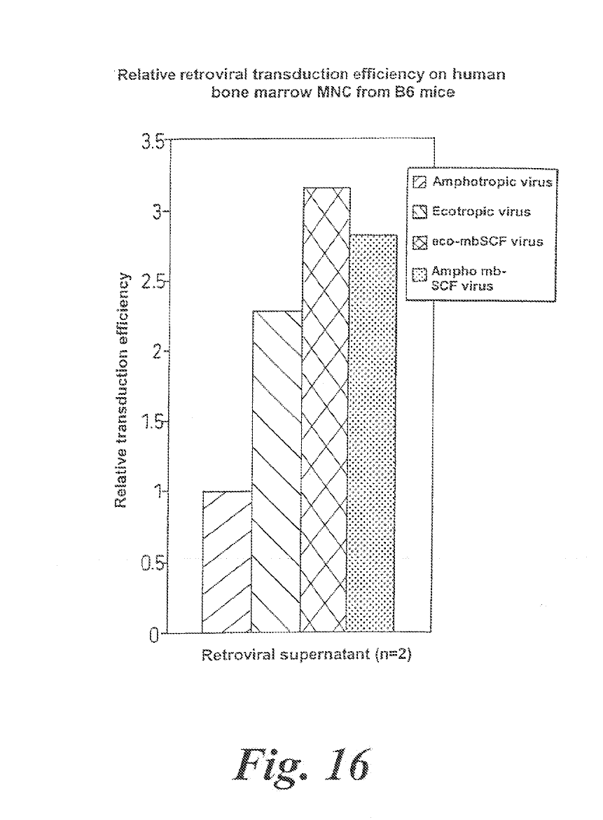

FIG. 16 Relative transduction efficiency of SCF—modified ecotropic virus on mouse bone marrow cells.

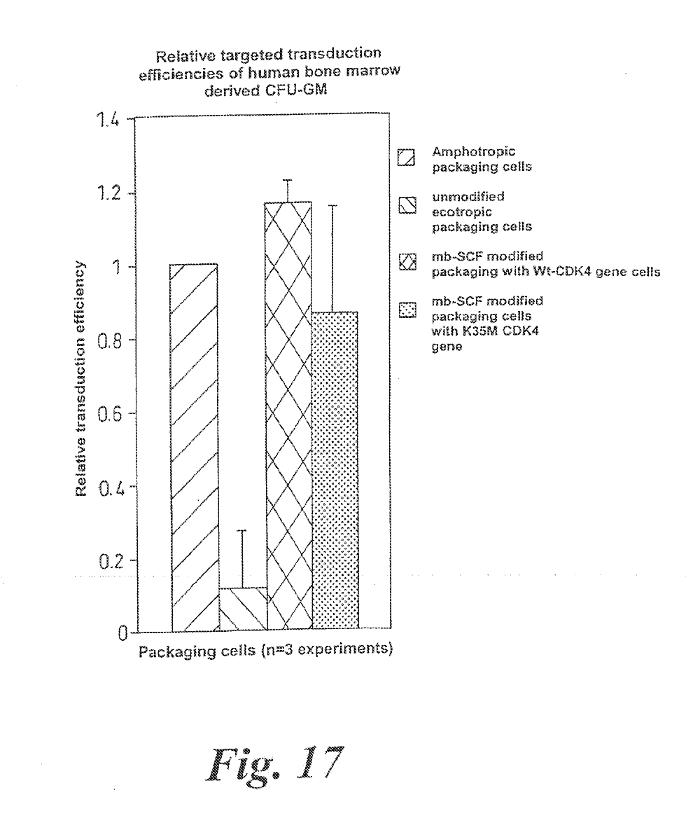

FIG. 17 Relative targeted transduction efficiencies of human bone marrow derived CFU-GM

FIG. 18 Schematic diagram showing sites of budding of various enveloped viruses.

### EXAMPLE 1

### Cloning of the Human Membrane Bound SCF cDNA

This example demonstrates a method for cloning a human gene encoding a membrane bound polypeptide. Stem cell factor (SCF) is a ligand that binds to the c-kit receptor protein found on the surface of quiescent stem cells (Hamel and Westphal, 1997). SCF exists in two forms, a longer soluble form and a shorter membrane bound form. The two forms results from differential mRNA splicing, with the soluble form having a proteolytic cleavage site coded by exon 6, while the membrane bound form has no exon 6 and thus no proteolytic cleavage site (Hamel and Westphal, 1997). We wished to express the membrane bound SCF on the surface of a packaging cell line as a first step to inserting the ligand into the envelope of a viral particle.

### 1. A SCF-Expressing Human Cell Line

The human stromal cell line, L88.5, a cell line expressing both the membrane bound form and the soluble form of stem cell factor (SCF) is derived from simian virus 40 (SV40)-transformed human stroma grown from the bone marrow of a haematologically normal patient (Thalmeier et al., 1994). It expresses both isoforms of SCF (the soluble isoform at a higher ratio then the membrane-bound isoform), and was used as the source of membrane-bound SCF (mb-SCF) for cloning. It was routinely maintained in Dulbecco's Modified Eagle's Medium (DMEM) supplemented with 10% heat inactivated Fetal Calf Serum (FCS; Gibco). L88.5 was a kind gift from Dr Karin Thalmeier (Germany).

Cell lines were grown at 37° C. in humidified 5% CO₂ in air in the incubator (New Brunswick Scientific, UK). Cells grown in suspension were maintained at a density of 2-5×10⁵ cells/ml. Adherent cells were passaged at 70-80% confluence: cells were detached from the tissue culture flask on incubation with trypsin-EDTA at 37° C., 5% CO₂ in air for 2 minutes, centrifuged at 1200 rpm for 5 minutes and resuspended in the appropriate growth medium.

2. Isolation of Total RNA from Human Cell Line L88.5

Total RNA was extracted from human cell line L88.5 cells when the cells were sub confluent. Solutions used in RNA manipulation were treated with the RNAase inhibitor diethyl pyrocarbonate (DEPC). Solutions containing 0.1% DEPC were left at room temperature for 12-16 hours Glassware was soaked in DEPC-ddH₂0 for 2 h, rinsed several times with ddH₂0, and autoclaved. DNAse and RNAse free (manufacturer certified) tips and plastic ware were used for all RNA preparation and never reused. Electrophoresis tanks were cleaned with detergent, rinsed in ddH₂0, dried with ethanol, soaked in 3% H₂O₂ and rinsed thoroughly in DEPC-treated ddH₂0. Gloves were worn at all times. RNA extraction was performed using dedicated pipettes. Total RNA was extracted from approximately 5×10⁶ cells under RNAase-free conditions. Briefly, cells were directly lysed in 0.2 ml RNAzol™ B (Biogenesis Limited, Bournemouth, U.K.) per 10⁶ cells. RNA was extracted by the addition of 10% v/v chloroform. Samples were shaken vigorously for 15 seconds, incubated on ice for 5 minutes and centrifuged at 13000 rpm, at 4° C. for 15 minutes. The colourless aqueous upper phase containing RNA was removed and RNA was precipitated by the addition of an equal volume of isopropanol, incubation at 4° C. for 15 mM and centrifugation at 13000 rpm, 4° C. for 15 minutes The RNA pellet was washed in 75% ethanol, and re-pelleted by centrifugation at 7500 rpm at 4° C. for 15 minutes. The pellet was air dried for 10-15 minutes, and dissolved in sterile DEPC-treated ddH₂O. The concentration and purity of total RNA was quantified by optical density (OD) measurement at wavelengths of 260 nm and 280 nm. An OD of 1 unit (U) corresponds to approximately 40 μg/ml of RNA. OD_{260/280} ratios of 1.8 and 2.0 indicate high purities of DNA and RNA respectively. The integrity of extracted RNA was determined by spectrophotometry and agarose gel electrophoresis. The A_{260/280} ratio was consistently found to be 2.0 from three different experiments, and all the three species of RNA (28S, 18S and 5S) were detectable by 0.8% agarose gel electrophoresis.

3. RT-PCR Amplification of the Membrane Bound-Scf mRNA

First strand cDNA was then made from the total RNA preparation described above. One μg of total RNA was used as a template to produce cDNA. A reverse transcription reaction consisting of 1 μg RNA, 10 mM of dNTP stock, 100 pmol/μl Random hexameres, 5×RT (Ist strand) buffer, 0.005M DTT and 2 U MMLV reverse transcriptase (Gibco, UK) was incubated at 37° C. for 1 hour. The reaction was subsequently heated to 95° C. for 5 mM to denature the RT enzyme. The reaction product was held on ice until used or stored long-term at −20° C.

The cDNA derived from the L88.5 cell line was used as template DNA to clone the membrane-bound SCF by PCR using SCF primers MF and MR. FIG. 1 is a schematic representation of the position of the primers used. The primer sequences used are:

| | |
|---|---|
| SCF MF | ACACCACTGTTTGTGCTGGATC |
| SCF MR | AACTGTTACCAGCCAATGTACG |

The amplification reaction consisted of 2 μl cDNA, 1 μM SCF forward and reverse primers 200 μM dATP, dCTP, dGTP, dTTP (Sigma), 1× reaction buffer containing 1 mM Tris-HCl (pH 9.0 at 25° C.), 5 mM KCl, 0.01% Triton®X-100), 1.5 mM MgCl₂ (Sigma) and 1.25 U Taq DNA polymerase (Sigma) in a total reaction volume of 50 μl. Hot start PCR was carried out by adding the Taq polymerase enzyme after the first 10-15 seconds of the initial denaturing step at 94° C. The annealing temperature was optimised to 60° C. for 1 minute and the extension temperature to 72° C. for 1 minute. Thirty four cycles were carried out with the final cycle having an increased extension time of 10 minutes. PCR products were electrophoretically analysed in a 2% agarose gel containing 0.5 μg/ml ethidium bromide. DNA was visualised by illumination with shortwave UV radiation (300 nm) on a Foto UV 300 series transilluminator (Fotodyne Incorporated), and photographed on Polaroid 667 film (Polaroid U.K. Limited).

Three distinct bands were observed on a 2% agarose gel in three different experiments, including fragments with sizes of 913 bp (giving rise to the soluble SCF), 829 bp (the membrane-bound-SCF) and an unidentified fragment of about 700 bp. As a positive control for the RT-PCR, GAPDH 5′ and 3′ primers were used, which gave the predicted size fragment of 350 bp. The GAPDH primer sequences are shown below.

| | |
|---|---|
| GAPDH 5′ | GTGAAGGTCGGAGTCAACG |
| GAPDH 3′ | GAGATGATGACCCTTTTGGC |

Negative controls were performed by substituting template DNA with water and by omitting the reverse transcriptase enzyme in the RT reaction, using the product as template for the PCR reaction. The results of the RT-PCR reaction are shown in FIG. 2.

4. Cloning of the mb-SCF cDNA

PCR-amplified SCF fragments were separated by agarose gel electrophoresis (as described above) and visualised by illumination under shortwave UV radiation (300 nm). The 829 bp fragment corresponding to the membrane-bound SCF was excised with a scalpel. The mb-SCF DNA fragment was gel purified using the Advantage™ PCR-Pure Kit (Clontech Laboratories—distributed by Cambridge Bioscience, Cambridge, U.K.) according to the manufacturer's instructions. The supernatant containing the purified mb-SCF cDNA was transferred to a fresh tube and the yield and purity measured by spectrophotometry. Solutions of purified DNA fragments were stored at −20° C.

Ligation of the gel purified mb-SCF fragment DNA with linearised pCR2.1 cloning vector was performed using a 1:3 molar ratio of linearised pCR2.1 vector (50 ng):gel purified mb-SCF cDNA fragment (insert DNA). Vector and insert DNA were incubated at 14° C. for 14-16 h in the presence of 1× ligase reaction buffer (Clontech, UK): 30 mM Tris-HCl pH 7.8, 10 mM MgCl₂, 10 mM DTT, 0.5 mM ATP) and 2 U T4 DNA ligase (Promega) in a total volume of 10 μl. Positive and negative control ligation reactions comprising manufacturers control plasmid were performed in parallel.

The pCR2.1 cloning plasmid was obtained from Invitrogen (Netherlands) and was used with the TA cloning kit (Invitrogen) for all PCR cloning. The TA Cloning Kit Dual Promoter with pCR II (vector) provided a quick, one-step cloning strategy for the direct insertion of a PCR (or RT-PCR) product into a plasmid vector. The principle of TA cloning is based on the fact that Taq polymerase has a non-template dependent activity that adds a single deoxyadenosine (A) to the 3′ ends of PCR products. The linearised plasmid vector (pCR2.1) supplied in the kit has single 3′ deoxythymidine (T) residues. This allows PCR inserts to ligate efficiently with the vector

The above ligation reaction was transformed into competent INV Fa strain of E. coli (Invitrogen). Frozen competent E. coli cells were thawed on ice. Three μl of the ligation mixture was added to one vial of competent cells and incubated on ice for 30 minutes. Cells were subjected to heat shock at 42° C. for 30 seconds and immediately placed on ice for 2 minutes. 250 μl of SOC medium was then added, and the transformation mixture incubated at 37° C. for 60 minutes with shaking at 225 rpm. The total volume was plated onto 15% Luria Bertani (LB) agar plates containing 100 μg/ml ampicillin and the plates were incubated at 37° C. for 14-16 hours. Positive and negative control transformation reactions containing closed circular plasmid DNA and linear DNA or ddH₂0 were performed in parallel. Transformation efficiency was calculated by performing a test transformation with closed circular plasmid DNA. The number of colonies produced using 1, 10 and 10 ng DNA provides an estimation of colony forming units/μg.

Screening bacterial colonies for recombinant transformants on the basis of β-galactosidase activity is possible if the insert DNA is cloned into an appropriate lacZ-peptide-coding vector and transformed in the INV Fα strain of E. coli. Appropriate vectors carry a fragment of the E. coli lac operon, coding for the amino-terminal fragment of the β-galactosidase enzyme. The INV Fα strain carries a lac l^{q}ZΔM15 mutation complemented by intra-allelic-complementation in the presence of the lacZ-peptide. Complementation is indicated by the formation of blue colonies in the presence of the β-galactosidase substrate 5-bromo-4-chloro-3-indolyl-β-_{D}-galacto-pyranoside (X-gal). DNA cloned into a multiple cloning site within the β-galactosidase coding sequence of the pCR2.1 cloning vector causes insertional inactivation, resulting in loss of β-galactosidase activity as indicated by the production of white colonies. Briefly, LB agar plates containing 100 μg/ml ampicillin (amp) were pre-coated with 40 μl of 20 mg/ml X-gal (Boehringer Mannheim) and 4 μl of 200 mg/ml (800 μg) IPTG and allowed to dry for 2-3 hours at 37° C. One hundred μl of the above transformation mix was plated and incubated for 12-16 hours. at 37° C. The resulting white colonies containing the mb-SCF cDNA were screened by restriction digest analysis and PCR using SCF primers MF and MR as above, resulting in an amplification fragment of 829 bp.

The orientation of the fragments in the pCR2.1 vector was also initially determined by PCR using M13-specific forward primers (on the flanking pCR2.1 sites) and SCF-specific primers (MR) (data not shown) and was confirmed by sequencing (Advanced Biotechnology Centre, ICSM Charing Cross Campus) (sequence not shown). The initial clone selected for sequencing revealed a Taq induced error (sequence not shown). Three more clones were then selected and sequenced. Clone 2 had the correct sequence of mb-SCF and was selected for sub-cloning experiments.

5. Cloning of the mb-SCF cDNA into a Mammalian Expression Vector

The pCR2.1 mb-SCF clones were sequenced at The Advanced Biotechnology Centre, Charring Cross Hospital. One clone containing the correct mb-SCF sequence and orientation (clone 2) was used to further sub-clone the mb-SCF insert into the pREP8 expression plasmid. The pREP 8 mammalian expression plasmid was obtained from Invitrogen. This plasmid was designed for high level, constitutive expression of DNA products utilising an RSV promoter. It has a histidinol resistance gene as a selectable marker for the expression of the desired cloned gene. This expression plasmid also containes the Epstein Barr Virus (EBV) Origin of Replication (oriP) and Epstein Barr Nuclear Antigen-1 (EBNA-1) genes enabling the plasmid to replicate as an episome.

Plasmid DNA from pCRII.1 mb-SCF clone 2 and pREP8 bacterial cell lines was prepared by growing the bacterial strains in liquid culture. A sterile culture of medium containing the 100 μg/ml amp was inoculated with bacteria picked from a single, freshly grown colony and incubated for 12-16 hours. at 37° C. with shaking at 220 cycles/minutes. Subsequent purification of plasmid DNA from the bacterial culture was performed using the Qiagen MinipREPs™ DNA Purification System (Qiagen), following the manufacturer's instructions. The protocol involved isolation of plasmid DNA by alkaline lysis and subsequent purification by binding to a silica-based matrix. Briefly, 5 ml of bacterial culture was grown as described above and plasmid DNA was extracted according to manufacturer's instruction.

The recombinant pCRII.1 mb-SCF clone 2 and pREP8 were both digested with Not I and Hind III restriction enzymes. Digestion of plasmid DNA was carried out by incubation at 37° C. for 14 hours in the manufacturer's 1× digestion buffer in the presence of 1-2 U of restriction enzyme. Digestion was confirmed by agarose gel electrophoresis. The mb-SCF cDNA insert DNA in pCRII.1 mb-SCF clone 2 was isolated by electrophoresis of the restriction enzyme digestion reaction on an agarose gel and recovery of the insert DNA from the agarose gel as described above. The recovered mb-SCF cDNA was subsequently ligated into the linearised pREP8 vector using the same approach as described above. The ligation mixture was then transformed into transformation competent E. coli strain INV-F cells as described above. The resulting recombinant clones, designated pREP8 mb-SCF clones, were screened by PCR and mini pREPs to identify a recombinant clone containing a single copy of the mb-SCF cDNA inserted into the pREP8 vector in the correct orientation. One of these clones, termed pREP8 mb-SCF, was then selected and used further.

### EXAMPLE 2

### Expression of the mb-SCF cDNA in Retroviral Packaging Cells

A cDNA clone coding for the human mb-SCF polypeptide was isolated and cloned into a mammalian expression vector as described above. We wished to then express the mb-SCF on the surface of a packaging cell line as a next step to inserting the polypeptide into the envelope of a viral particle.

1. Transformation of a Viral Packaging Cell Line with pREP8 mb-SCF

Clone pREP8 mb-SCF was selected for transformation into a viral packaging cell line. This clone has a cDNA encoding the mb-SCF polypeptide inserted into a mammalian expression vector. As a first step, plasmid DNA was extracted from the clone using a large-scale plasmid purification procedure. Bacteria picked from a freshly grown colony were used to inoculate 5 ml of LB broth. The culture was incubated in the presence of the appropriate antibiotic for 12-16 hours. at 37° C. with shaking at 220 cycles/minute, and used to inoculate 300 ml LB broth (large-scale plasmid pREParation). The culture was incubated in the presence of the appropriate antibiotic for 12-16 hours at 37° C. with shaking at 300 cycles/minute The large-scale extraction of plasmid DNA was performed using the Qiagen MaxipREP™ DNA Purification system, according to the manufacturer's instructions.

Plasmid DNA was then used to transform the Phoenix ecotropic retroviral packaging cell line. Phoenix ecotropic and amphotropic packaging cell lines were kind gifts from Dr Gary Nolan, Stanford University, California, USA (Grignani et al., 1998; Kinsella and Nolan, 1996). These cell lines are second generation retrovirus-producer lines for the production of helper-free ecotropic and amphotropic retroviruses. The lines are based on the 293T cell line, a human embryonic kidney cell line. They produce gag-pol genes with hygromycin as a co-selectable marker, and envelope proteins (either ecotropic or amphotropic) with diphtheria toxin as a second selectable marker. These cell lines were routinely maintained in DMEM supplemented with 10% FCS. They were additionally passaged for one week, about every 10 to 12 weeks, in 300 μg/ml hygromycin and 1 μg/ml diphtheria toxin for reselection.

Transfection of the Phoenix ecotropic retroviral packaging cell line with recombinant plasmid pREP8 mb-SCF was performed by calcium phosphate precipitation. Briefly, 12 μg plasmid DNA in 500 μl 250 mM CaCl₂ was added dropwise to an equal volume of 2×HMS, while bubbling air through. The resulting solution was incubated at room temperature for 20-30 minutes, allowing a fine precipitate to form, then added dropwise to cells grown to 60% confluence in a 75 ml tissue culture flasks. The cells were maintained at 37° C., in 5% CO₂ in air for 24 hours before the calcium phosphate containing media was removed. The cells were washed twice with 10% culture medium, resuspended with fresh 10% culture medium and incubated for a further 24 hours at 37° C., in 5% CO₂ in air. Selection of stable transformants was carried out in the presence of 2.5 mM histidinol. Resistant cells were propagated by splitting 1:5 the contents of 80% confluent flasks. Cells were kept in selection for at least 6-8 weeks. Stable transfectants were cryopreserved for further experiments.

The above stable cells obtained after 6-8 weeks were further subjected to dilution cloning. Briefly, 500 cells were seeded in 100 μlof DMEM supplemented with 10% FCS and 2.5 mM histidinol, in a 96-well flat-bottom plate. Four wells that reached confluence within a week were trypsinised and transferred into 48 well plates containing 300 μl DMEM supplemented with 10% FCS and 2.5 mM histidinol for a further week. These clones were then propagated into T25 flasks containing 10 ml of DMEM supplemented with 10% FCS and 2.5 mM histidinol. The four individual clones were then subjected to further experiments to determine the presence and expression of the mb-SCF in these cells.

2. Detection of pREP8 mb-SCF Plasmid in the Transformed Phoenix Cell Lines

Retroviral packaging cells that were stably transfected with pREP8 mb-SCF plasmid were confirmed to contain the plasmid by PCR. Briefly, clones growing in T25 flasks containing DMEM supplemented with 10% FCS and 2.5 mM histidinol, were trypsinised. The trypsinised cells were washed twice with PBS and resuspended in 500 μl of PBS. Two microlitres of the cell suspension was used as a template in a standard hot start PCR (see above) containing pREPF and EBV R primers.

| | |
|---|---|
| PREP F | GCTCGATACAATAAACGCC |
| EBV R | GTGGTTTGTCCAAACTCATC |

PCR products were run on a 2% agarose gel containing 0.05% ethidium bromide. Positive controls for the amplification of the plasmid DNA were pREP8 mb-SCF DNA itself as template DNA. Negative controls involved the use of water or untransfected cells as template in a standard PCR. The detection of pREP8 mb-SCF in the packaging cells using primers pREPF and EBVR, gave rise to an amplification product of 1164 bp (FIG. 3).

3. Expression of mb-SCF Proteins on Engineered Retroviral Packaging Cells.

A standard indirect immunofluorescence assay was employed to detect the cell surface expression of SCF by the engineered packaging cells. The stably transfected packaging cells were grown on glass cover slips. When the cells were about 70% confluent, the cover slips were removed from the incubator and the cells fixed with methanol for 10 minutes at room temperature. The cells were then incubated with anti c-kit ligand/stem cell factor antibody (goat anti human SCF) (R&D Systems) at 1:5 dilution at 37° C. for 30 minutes. The cover slips were washed twice with PBS for 10 minutes and incubated with goat anti human anti IgG FITC conjugated secondary antibody. The cover slips were washed twice with PBS for 10 minutes and mounted onto a glass slide with 10% PBS/90% glycerol mountant and viewed under an Olympus UV microscope. Engineered packaging cells expressed high levels of SCF polypeptide as seen by intense fluorescent staining while non-engineered cells did not fluoresce (FIG. 4). Antibody specificity was also confirmed by the use of non-immune goat serum on engineered packaging cells, which did not give a signal. No differences were seen in the signal intensities and number of fluorescing cells among the four separate clones.

The four modified packaging cell clones arising from histidinol selection were also analysed by flow cytometry to demonstrate the presence of mb-SCF. Briefly, transfected and untransfected cells growing in a T25 flask were detached using cell disassociation buffer, enzyme-free, PBS based (Gibco). Cells were washed twice in PBS. These cells were then incubated with a 1:5 dilution of anti c-kit ligand/stem cell factor antibody (goat anti human SCF) (R&D Systems) for 40 minutes at 4° C. Cells were then washed twice in PBS and incubated with goat anti human anti IgG FITC conjugated secondary antibody for 30 minutes at 4° C. Cells were washed twice and fixed for 20 min at room temperature using 35 μl Leucoperm Reagent A (Serotec). Cells were resuspended in 1 ml of PBS for flow cytometry analysis. Flow cytometric analysis was carried out in the Stem Cell Lab, Hammersmith Hospital, using a Beckman FACScan machine. The software used to acquire and analyse the data was Cellquest. Forward and side scatter properties of untransfected cells were determined, and fluorescence intensities of transfected cells compared to untransfected cells stained with the both the primary and secondary antibodies. On this analysis, the four clones appeared to have slight differences in the frequency of cells expressing mb-SCF ranging from 73-98%. The expression levels of mb-SCF as determined by the peak on the fluorescence axis were the same for all four clones. The highest mb-SCF expressing clone, Clone 3, which had 98% expression of mb-SCF was selected for retroviral production and is shown in FIG. 5. This clone was also confirmed to have high levels (97%) surface expression of CD59 as also shown in FIG. 5.

### EXAMPLE 3

### Production of Retroviruses from Engineered Retroviral Packaging Cells Containing mb-SCF as Part of the Viral Envelope

A cDNA clone coding for the human mb-SCF polypeptide was isolated and cloned into a mammalian expression vector as described above. We then expressed the mb-SCF on the surface of a packaging cell line. As a next step we wished to determine whether viral particles derived from the retroviral packaging cell line contained the mb-SCF as part of the viral envelope.

1. Production of Retroviruses from the Engineered Phoenix Cells

The Phoenix ecotropic cell line, the mb-SCF modified ecotropic Clone 3 and the amphotropic packaging cell lines were subsequently calcium phosphate transfected with the PINCO plasmid vector encoding the viral LTR, the packaging signal and a reporter gene, enhanced green fluorescence protein (eGFP) (Grignani et al., 1998). The PINCO vector contains the full length Moloney murine leukaemia virus LTRs, the extended ψ sequence and a transcriptional cassette composed of the CMV promoter and the cDNA for the Enhanced Green Fluorescent Protein (eGFP) In addition to the retroviral backbone, this vector also contained the EBV oriP and EBNA-1 gene enabling the vector to replicate as an episome. This vector was also a kind gift from Dr Gary Nolan (Stanford University, Leyland, USA). Transient retrovirus supernatant was obtained prior to puromycin (1 μg/ml) selection. Following the removal of the calcium phosphate-DNA mixture and the overnight recovery of the packaging cells in complete medium, the medium was removed and replaced with 5 ml of fresh medium and the cells were incubated at 37° C. Virus-laden supernatant was collected after 24 hours, 0.45μ filter sterilised, aliquoted and stored at −70° C. The PINCO transfected cells were propagated for a further 6 weeks in selection conditions with 1 μg/ml puromycin. Retrovirus was harvested in the same way as the transient supernatant.

2. Calculation of Viral Titre from mb-SCF Expressing Cells

Expression of the eGFP reporter gene was analysed by flow cytometry analysis of transduced NIH3T3 cells to obtain the retroviral titre. These cells were already available in the laboratory and were used to determine retroviral titres. They are maintained in DMEM supplemented with 10% FCS. Retroviral titres from transient and stable transfection of the PINCO vector into the three retroviral packaging cell lines (Phoenix Amphotropic, Ecotropic and Mb-SCF modified Ecotropic) were determined on NIH 3T3 cells. NIH 3T3 (5×10⁴) cells were seeded in a 24 well plate. One ml of an infection cocktail, containing retroviral supernatants ranging from 1 μl to 100 μl, DMEM supplemented with 10% heat inactivated FCS and 8 μg/ml polybrene, was added to individual wells. Infection was carried out overnight at 37° C. The following day, the infection cocktail was removed and 1 ml of fresh medim was added. Incubation was carried out at 37° C. for a further 48 hours. The cells were then trypsinsed, washed twice in PBS, and fixed with Leucoperm. The cells were than flow cytometrically analysed to determine the expression of eGFP in the NIH 3T3 cells. Scatter properties of uninfected NIH 3T3 cells were determined and retrovirally transduced cells were compared for fluorescence intensities. The titre was calculated by titration of 1, 10 and 100 μl viral supernatant (each in duplicate) in a total volume of 1 ml of infection cocktail (containing complete medium and 8 μg/ml polybrene). The positive cell frequency measured as the percentage of fluorescent cells was plotted against the volume of supernatant used. The slope of the graph represented the retroviral titre. Retroviral viral titres are expressed as infectious particles (IP)/ml.

The retroviral titre of the mb-SCF engineered packaging cells are listed in Table 1. Retroviral titres between the unmodified ecotropic packaging cells and the mb-SCF modified packaging cells (stables) were not significantly different (FIG. 6).

TABLE 1

| | Retroviral titre on NIH 3T3 cells |
|---|---|
| Packaging cell Line | (IP/ml ± sd) (n = 3 experiments) |
| Phoenix Ecotropic | 3.88 ± 1.18 × 10⁵ |
| Phoenix mb-SCF modified | 2.98 ± 0.32 × 10⁴ |
| (transient) | |
| Phoenix mb-SCF modified | 2.60 ± 0.53 × 10⁵ |
| (stable) | |
| Phoenix Amphotropic | 2.08 ± 1.29 × 10⁵ |

3. Detection of mb-SCF Protein in Retroviral Particles Derived from mb-SCF Expressing Cells

Retroviruses were produced transiently and stably from the engineered ecotropic packaging cell, Clone 3, non-engineered ecotropic packaging cells and amphotropic packaging cells using the PINCO retroviral vector plasmid. Retroviral supernatant was collected from transiently transfected cells 48 hours post transfection with the PINCO plasmid and from stable transfectants that had been selected for 6 weeks in puromycin-containing medium. Four ml of these viral supernatants were pelleted by ultracentrifugation at 30,000 rpm for 1.5 h at 4° C. in a SW 55 Ti rotor (Beckman). The supernatant was discarded and the pelleted retroviral particles were resuspended in 50 μl of PBS and stored at −70° C.

The pelleted retrovirus particle protein concentration was determined by an assay similar to the well documented Lowry assay, using a commercially available kit (Bio-Rad DC Protein Assay (Bio-Rad)). Protein standards consisting of bovine serum albumin in the range of 0.2 mg/ml to 2 mg/ml were pREPared. Five microlitres of protein standards and samples (retroviral particles) were then assayed according to manufacterer's instructions using a Microplate Assay protocol. Protein concentrations of retroviral particles and protein standards were determined by absorbance at 750 nm.

Equal amounts of proteins were loaded gels for the detection of mb-SCF and CD59 in the particles by western blot analysis. 10 μg of retroviral particles were mixed in 2× gel loading buffer (containing 50 mM Tris Cl, pH6.8, 2% SDS, 10% glycerol and 1 mg bromophenol blue), 0.1 M dithiotreitol and boiled for 5 minutes at 95° C. This protein extract was then separated on a 12% polyacrylamide gel under reducing conditions. The protein extracts were electrophoresed in parallel with prestained protein markers in 1× running buffer (25 mM Tris pH8.3), 250 mM glycine and 0.1% SDS) at 80 V for about 3-4 hours until the bromophenol marker was about 1 cm from the bottom of the gel. The proteins were transferred to a nitrocellulose membrane (Immobilon) using a semi-dry transblotting apparatus (Bio-Rad). One×transfer buffer was the same as the 1× running buffer but contained 20% methanol. The transfer of proteins was carried out according to manufacturer's instructions, running at a constant current of 0.8 mA/cm² for 1 hour. The success of transfer was determined by the detection of the prestained protein markers on the membrane, and the positions of these protein standards were marked with a pencil. The membrane was blocked with 10% milk protein (Marvel) in 1×TBST (Tris Buffered Saline and Tween20) for 1 hour at room temperature with gentle agitation. The demonstration of SCF on the retroviral particles was carried out by incubating, the membrane with goat anti-human SCF antibody (R&D Systems) at 1:500 dilution in 1×TBST containing 10% Marvel, at 4° C. for 18 hours. Human CD59 proteins on retroviral particles were demonstrated by incubating the membrane with mouse anti-human CD59 antibody (Serotec) at 1:250 dilution in 1×TBST containing 10% Marvel, at 4° C. for 24 hours. Antibody-treated membranes were then washed 3 times for 20 minutes in copious volumes of 1×TBST and a final wash for 20 minutes with 1×TBS. The SCF and CD59 antibodies were then detected by incubation with secondary donkey anti-goat IgG horse radish peroxidase (HRP) at a dilution of 1:2500 for 1 hour in 1×TBST containing 10% Marvel; and at a dilution of 1:500 secondary chicken anti-mouse IgG HRP, respectively. Membranes were subsequently washed 3 times for 20 minutes in copious volumes of 1×TBST and a final wash for 20 minutes with 1×TBS. SCF and CD59 antibodies were visualised using an enhanced chemiluminescence's kit (Amersham) according to manufacturer's instructions. Membranes reacted with the appropriate substrates from the kit, were then subjected to autoradiography. Autoradiography was conducted using different exposure times, ranging from 30 sec to 5 minutes, to obtain the best signals. Exposed autoradiography films were developed using an automatic film developer.

Retroviruses produced following transient transfection and stable producers were found to express the engineered mb-SCF, with the transient supernatant appearing to have a slightly higher level of expression (FIG. 7). The non-engineered cells did not express the mb-SCF as expected. CD 59, a naturally occurring membrane protein in the packaging cells (engineered and non-engineered) could also be demonstrated.

### EXAMPLE 4

### Functionality of the mb-SCF Containing Viral Particles

We have demonstrated above that it is possible to insert a membrane-bound polypeptide expressed in a viral packaging cell line into the surface envelopes of virus particles derived from that cell line. We now wished to determine whether the incorporated polypeptide retained biological activity. The mb-SCF polypeptide can bind to the c-kit receptor protein found at the cell surface of quiescent cells (Sehgal et al., 1999). Once bound it can induce the cells to proliferate (Sehgal et al., 1999). Hence we measured the capacity of the modified viral particles to bind to and induce proliferation of cell lines expressing the c-kit receptor.

1. Binding of the mb-SCF Viral Particles to C-Kit Receptor Positive Cells

To show that retroviral particles expressing mb-SCF could specifically bind to c-kit expressing cells, retrovirus binding experiments, as demonstrated by flow cytometry using the anti-SCF monoclonal antibody, were also carried out. The human megakaryoblastic leukaemia cell line, Mo7e, has been used in retroviral targeting experiments as the cell line has been reported to express high levels of c-kit (the receptor for SCF). Mo7e cells were maintained RPMI supplemented with 20% FCS and 10 ng/ml GM-CSF. Mo7e cells were incubated with 100 μl retroviral supernatant at 32° C. for 1 hr. Cells were washed twice in cold PBS. One hundred microlitres of monoclonal antibody, 83A25 were then incubated with cells at 4° C. for 1 hour. These cells were washed twice with cold PBS. Cells were finally incubated with a FITC-conjugated rat anti-mouse IgG antibody for 1 hour at 4° C. The cells were then washed twice in cold PBS and analysed by flow cytometry.

The mb-SCF retrovirus bound to the Mo7e cells whereas the unmodified ecotropic virus did not. Prior down regulation of the kit receptor by growing the Mo7e cells overnight in SCF instead of GM-CSF (confirmed by flow cytometry) abrogated binding of the mb-SCF retrovirus (FIG. 8). These experiments demonstrate that targeted retroviral binding was achieved, resulting from the modification of the packaging cells.

### 2. Mo7e Cell Proliferation Following Exposure to the Modified Viral Particles.

The biological activity of retroviral particles containing the mb-SCF proteins was demonstrated using a standard commercially available proliferation assay, CellTiter 96® AQᵤₑₒᵤₛ One Solution Cell Proliferation Assay (Promega). Mo7e cells were washed twice in PBS and resuspended in RPMI supplemented with 20% FCS. 5000 cells per 90 μl or per 70 μl of resuspended media, were dispensed into 96 well round bottom microtitire plates. Ten μl or 30 μl of retroviral supernatant (prepared as above) from the Phoenix ecotropic mb-SCF cell lines, was added to the wells of the 96-well round bottom plates to bring the total volume of cell suspension to 100 μl. Positive controls for the proliferation assay included the use of 100 ng/ml recombinant purified human SCF. Negative controls included no cytokines or viral supernatants from the Phoenix ecotropic cells. Eight identical plates (each concentration in triplicate) were set up, with the first plate being assayed for background proliferation activity after two hours of having being set up.

Proliferation was measured every 24 hours by the addition of 20 μl of a solution containing, tetrazolium compound; 3-(4,5-dimethylthiazol-2-yl)-5-(3-carboxymethoxyphenyl)-2-(4-sulfophenyl)-2H-tetrazolium, inner salt (MTS) and an electron coupling reagent, phenazine ethosulfate (PES) to each well, and incubation for 1.5 hours, at 37° C. in a humidified incubator. The MTS compound is bioreduced by cells into a coloured formazan product that is soluble in tissue culture medium. The colour reaction was quantified by recording the absorbance at 490 nm with a 96-well plate reader.

Viral particles from mb-SCF engineered paakaging cells were able to stimulate the proliferation of Mo7e cells. Control retroviral particles from unmodified ecotropic packaging cells and cells incubated without the addition of cytokines did not stimualte the Mo7e cells. It is intresting to note that the proliferation from the mb-SCF modified retrovirus begun only after day 3 of the experiment. Increasing the volume (to 30 μl) of retroviral supernatant did not appear to increase the rate of proliferation of the Mo7e cells. Retrovirus obtained transiently, was also capable of stimulating the proliferation of Mo7e cells. The positive control for the expriment was 100 ng/ml recombinant human SCF, which as expected stimulated the Mo7e cells. The results are plotted in FIG. 9.

### EXAMPLE 5

### Gene Transfer Using Viral Particles of the Invention and eGFP as a Reporter Gene

The examples above demonstrate that the methods of the invention can be used to incorporate a biologically active passenger peptide binding moiety into a viral particle. Here we show that such viral particles are capable of transducing target cells bound by the passenger peptide binding moiety.

### Methods

### Retroviral Vector Plasmid, PINCO

The retroviral vector used to generate retrovirus from the Phoenix retroviral packaging cells (amphotropic and ecotropic) was called PINCO (Grignani et al., 1998; Kinsella and Nolan, 1996). The PINCO vector contains the full length Moloney LTRs, the extended ψ sequence and a transcriptional cassette composed of the CMV promoter and the cDNA for the Enhanced Green Fluorescent Protein (eGFP). In addition to the retroviral backbone, this vector also contained the EBV oriP and EBNA-1 gene enabling the vector to replicate as an episome. This vector was also a kind gift from Dr Gary Nolan (Stanford University, Leyland, USA).

### Methods

### Primary Cells

### Normal Peripheral Blood Progenitor Cells (PBPCs)

G-CSF mobilised peripheral blood from normal individuals obtained from the Stem Cell Laboratory, Hammersmith Hospital. These cells were acquired with signed informed consent and research ethics committee approval.

### Murine NIH 3T3 Cell Line

These cells were already available in the laboratory and were used to determine retroviral titres. They also were maintained in DMEM supplemented with 10% FCS.

### Human Erythroleukaemia Cell Line TF-1 and Human Megakaryoblastic Leukaemia Cell Lines LAMA-84 and Mo7e

The TF-1 cell line, a human erythroleukaemia cell line was maintained in RPMI supplemented with 10% FCS and 1 ng/ml GM-CSF (Kitamura et al., 1989). The human megakaryoblastic leukaemia cell line, Mo7e, was maintained RPMI supplemented with 20% FCS and 10 ng/ml GM-CSF. The human megakaryoblastic leukaemia cell line, LAMA-84, was maintained in RPMI supplemented with 10% FCS. These cell lines were used in retroviral targeting experiments as they have been reported to express high levels of c-kit (the receptor for SCF). The Mo7e cell line was also used in a proliferation assay to demonstrate biological activity of mb-SCF in retroviral particles. The Mo7e and LAMA-84 cell lines (Lee et al., 2001; Miyazawa et al., 1995; Seigneurin et al., 1987) were kind gifts from Dr. Junia Melo. The TF-1 cell line was available in the laboratory.

### Supernatant Transduction of Haematopoietic Cell Lines

Multiplicity of infection (MOI) was calculated based on the retroviral titre on NIH3T3 cells and retroviral targeting experiments were carried out with an MOI of at least 2. An MOI of 1 represents one infectious retroviral particle per target cell. An infection cocktail containing retrovirus, 10% FCS in DMEM and 8 μg/ml polybrene was incubated with 25,000 target cells for 24 hours at 37° C. at 5% CO₂ in air. The medium was removed and replaced with fresh medium and incubated for a further 48 hours at 37° C. at 5% CO₂ in air. Target cells were then washed twice with PBS, fixed and assayed by flow cytometry.

### Co-Culture of Haematopoietic Cell Lines

Transduction of non-adherent haematopoietic cell lines by co-culture was performed with a 2-fold excess of producer cells. Retroviral producer cells were grown to confluence, trypsinised and irradiated at 2000 rads. Cells were replated into a 6-well microtitre plate, maintaining confluence, and incubated at 37° C. in 5% CO₂ in air for 16-18 h. Cells were washed carefully in PBS and target cells (TF-1) in log-phase growth (5×10⁴) were added in a total volume of 2 ml. A mock transduction in the absence of target cells was performed in parallel to control for residual producer cell activity. Cells were incubated at 37° C. in 5% CO₂ in air for 24 hours. Non-adherent cells were removed, placed into a fresh 6-well microtitre plates and incubated for a further 8 h at 37° C., 5% CO₂ in air in order to facilitate adherence of contaminating producer cells prior to assay of vector reporter genes. Non-adherent cells were then resuspended in fresh medium and incubation carried out for a further 48 hours at 37° C., 5% CO₂ in air. Target cells were then washed twice with PBS, fixed and assayed by flow cytometric analysis.

### Retroviral Transduction of Primary Cells

### Separation of Mononuclear Cells

Bone marrow cellsin preservative free heparin were diluted 1:4 in Hank's Buffered Salt Solution (Gibco). The diluted sample were layered over Lymphoprep (Nyegaard) and centrifuged at 1800 rpm for 30 minutes. Mononuclear cells (MNC) were removed from the interface and washed twice by centrifugation at 1800 rpm for 10 minutes in Hank's Buffered Salt Solution. MNC were counted using a haemocytometer chamber following dilution in 3% acetic acid to lyse any contaminating erythrocytes.

### CD 34+ Cell Separation

CD34+ cells were separated from MNC using the MiniMacs™ system (Miltenyi Biotec) according to manufacterer's instructions. Briefly, MNCs were resuspended in a buffer containing of calcium- and magnesium-free PBS with 0.5% human serum albumin (Immuno AG) and 5 mM EDTA at 500 μl per 10⁸ MNC. One hundred μl each of reagants A1 (a blocking IgG) and A2 (QBEND/10-a mouse IgG recognising CD34) was added to the cells followed by incubation for 15 minutes at 4° C. Cells were washed in 5 ml of buffer and resusupended in 400 μl of buffer per 10⁸ MNC. One hundred μl of reagent B (colloidal superparamagnetic MACS microbeads recognising reagent A2) was added to the cells and incubated for 15 minutes at 4° C. followed by another wash as above. The cells were resuspended in 500 μl of buffer then added to a prefilled MiniMacs™ column (type MS) held in a magnet and the cells allowed to pass through. Labelled CD34+ cells are retained in the column by the magnet. These cells are washed 4 times by adding 500 μl of buffer and allowing it to pass through. The column was removed from the magnet and the positive cells were eluted with 1 ml buffer. These cells were enumarated using a haemocytometer and the purity determined by staining a sample of cells with HPCA2 antibody (Beckton Dickinson), recognising a different epitope of the CD34 molecule, conjugated with FITC and FACS analysed. The purity of the samples was consistently above 90%.

### Retroviral Transduction of CD34+Primary Cells Using the Transwell Technique

The day before starting transduction of the CD34+ cells, the stable AM-12 retroviral producing cell line containing the amphotropic retroviral envelope and the retroviral genome (pBabeNeo; Morgenstern and Land, 1990), was plated in a 6-well plate at a concentration of 2×10⁵ cells/well and maintained in DMEM supplemented with 10% FCS. On the day of the transduction, the medium of the producer cell line was replaced with 2.5 ml of fresh DMEM supplemented with 10% FCS and protamine sulphate at a concentration of 8 μg/ml which would finally equilibrate to 4 μg/ml after the addition of the transwell. One 24 mm Transwell with a 0.4 μm membrane (Costar) was inserted into each well. CD 34+ (10⁵) purified cells was resuspended in 2.5 ml of 30% FCS (Mycoplex™ PAA) and cytokine mix (50 ng/ml of IL-3, 100 ng/ml of SCF, 10 ng/ml of GM-CSF) in Iscoves Modified Dulbecco's Eagles Medium (Gibco) and transferred into the transwell inserts. The transduction process was carried out for 48 hours before the cells were harvested and cultured in semi-solid medium (methylcellulose), with and without G418 selection, for the growth of CFU-GM as described below.

### Clonogenic Assay of Retrovirally Transduced Progenitor Cells

### CFU-GM Assay

The CD34+ transduced cells were removed from the transwell and washed in Hank's Buffered Saline Solution. These cells were cultured in 3 ml methylcellulose (MC, Methocult H4230, Stem Cell Technologies) supplemented with 300 μl of CFU-GM mix (containing 50 ng/ml SCF, 1 ng/ml GM-CSF, 10 ng/ml IL-3 and 100 ng/ml G-CSF) and 1.5 mg/ml G418. After the cells were mixed well in MC, they were plated in 35 mm diameter petri dishes (Nunclon®) (1 ml/dish). The dishes in turn were placed in a 150 mm bacteriological petri dish (Sterilin) along with 35 mm petri dishes without lids containing sterile water to reduce the loss of culture medium through evaporation. The cultures were incubated at 37° C. in humidified 5% CO₂ in air. CFU-GM colonies of more than 50 cells, were scored on day 7 of incubation using a Leica DM1L inverted microscope.

### Evaluation of Gene Transfer to Primary Cells

### Transduction Efficiency

The efficiency of each CD34+ transduction experiment was calculated from the proportion of surviving CFU-GM colonies after 14 days of incubation in culture with G418 at a concentration of 1.5 mg/ml as described earlier.

$$Transduction efficiency = \frac{No . of colonies surviving in G 418}{No . of colonies without G 418} \times 100$$

### Results and Discussion

Experimental data is presented in FIGS. 11 to 17. All data have been expressed as efficiency of transduction relative to that obtained from the same viral vector packaged in an equivalent amphotropic packaging cell line i.e. Phoenix amphotropic packaging cells.

FIGS. 11 to 14 present data from viral particle transduction of three different SCF-receptor (kit) positive cell lines (Mole (FIG. 11), TF-1 (FIG. 12), LAMA (FIG. 13)) and one kit negative (U937, FIG. 14) cell line. Viral particles incorporating mb-SCF (labelled ‘mb-SCF’ in FIGS. 11 to 14) were prepared using the methods outlined in the above Examples. Ecotropic virus can only infect mouse cells, while amphotropic virus can infect both mouse and human cells.

As can be seen from FIGS. 11 to 14, viral particle transduction efficiency with unmodified ecotropic virus is very low for both kit positive and negative human cells. In contrast, transduction with ecotropic virus incorporating mb-SCF is highly efficient (similar efficiency to amphotropic), when target cells express high levels of kit (Mole and TF1 cells are >95%+ve for the presence of kit; FIGS. 11 and 12), somewhat less so when the kit expression is lower (LAMA cells are ˜50% for the presence of kit+ve; FIG. 13) and no different from unmodified ecotropic virus on kit negative cells (U937 cells; mb-SCF results labelled ‘C13 virus’; FIG. 14).

To demonstrate that the failure of the SCF-displaying viruses to transduce kit-negative cells is not a general defect of the ability of the viral particles to transduce these cells, data from experiments using amphotropic mbSCF-incorporating viral particles is shown as a control. Amphotropic mbSCF viral particles have a similar efficiency of transduction to unmodified amphotropic virus, and are capable of transducing kit negative cells (FIG. 14). Hence the modified viral particles incorporating the mb-SCF passenger binding motif are as capable of infecting susceptible cell types as normal viral particles.

Taken together, the data demonstrates that ecotropic viral particles incorporating the mb-SCF passenger peptide binding motif can transduce cells that have a receptor to which the mb-SCF peptide can bind. In contrast, ecotropic viral particles without the passenger peptide cannot transduce such cells. Hence, the method of the invention allows for viral particles to be produced that incorporate a passenger peptide binding motif that is capable of mediating target cell viral transduction. Such viral particles can be successfully used as a means for gene transfer to target cells.

FIGS. 15 to 17 present data from the viral particle transduction of human bone marrow derived primary haematopoietic stem cells. Viral particles types are as those used in the experiments presented in FIGS. 11 to 14.

The transduction rate of human bone marrow derived primary haematopoietic stem cells (CD34+ cells; FIG. 15) using viral particles incorporating the mb-SCF passenger peptide binding motif is equally, if not more, efficient than with amphotropic virus, whereas unmodified ecotropic virus, as expected, is very poor. Again, amphotropic mb-SCF-incorporating viral particles is highly efficient in this context, suggesting that the incorporation of mb-SCF on the virus can enhance transduction efficiency over conventional unmodified amphotropic virus.

FIG. 16 shows transduction of mouse bone marrow as a control. Here, we expect all viruses to transduce the target cells, and indeed, the ecotropic virus (specific for murine cells) is now superior to the amphotropic virus in efficiency. Both ecotropic mb-SCF and amphotropic mb-SCF viral particles, however, show enhanced levels of transduction, suggesting that the efficiency may be greater on kit positive cells, the receptor to which the mb-SCF passenger peptide binding motif binds. Human bone marrow derived primary haematopoietic stem cells will contain large numbers of cells with the kit receptor. It is worth noting that with the experiments with mouse cells there is no specific targeted transduction possible.

The experiments detailed above used the PINCO reteroviral vector which carries the enhanced green fluorescent protein (EGFP) gene and viral particle transduction of the cells was scored by analysis of fluorescence of cells using a FACs (fluorescence activated cell sorter).

Finally, FIG. 17 shows data from viral particle transduction of human bone marrow derived myeloid colony forming cells (CFU-GM). In these experiments the retroviral constructs carry a gene that confers resistance to the drug G418 (a derivative of the antibiotic neomycin, which can also kill mammalian cells). Bone marrow cell are transduced and then plated in semi-solid medium containing G418 to allow any resistant haematopoietic colonies to form. The colonies are scored after 14 days under an inverted microscope. Only successfully transduced cells should survive in the G418-medium to go on to produce colonies. The data are therefore derived from colony numbers obtained, following transduction with the different retroviruses and are again expressed relative to the performance of an equivalent amphotropic virus. The viral vector in these experiment was based on the pBabe vector (Morgenstern and Land, 1990).

As observed previously, unmodified ecotropic virus was very poor at transducing human haematopoietic progenitors—less than 10% of the rate or transduction mediated by amphotropic virus. In contrast, however, two different ecotropic mb-SCF constructs (labelled ‘CDK4 gene’ and ‘KL35M CDK4 gene’) gave highly efficient transduction of CFU-GM, a rate of transduction comparable to that observed using the amphotropic virus. Hence mb-SCF promotes ecotropic virus transduction of cells with the kit receptor.

These experiments confirm that viral particles incorporating the mb-SCF passenger peptide binding motif are capable of mediating target cell viral transduction. Such viral particles can be successfully used as a means for gene transfer to target cells.

### EXAMPLE 6

### Preparation of Viral Particles Containing More than One Engineered Polypeptides in the Viral Envelope

In Examples 1 and 2, a cDNA encoding a peptide to be incorporated into a viral particle was cloned from a human cell line and successfully expressed in a viral packaging cell line. Of course, it is possible to incorporate more than one peptide into a viral particle.

One a peptide to be used has been selected, for example, a peptide that is a ligand for a cell-type specific receptor, it is straightforward using the methods outlined in Example 1 to isolate a cDNA encoding the peptide from a population of cells in which the ligand is expressed. An alternative approach would be to conduct database searches to identify cDNAs encoding the ligand.

Once a cDNA encoding the ligand has been successfully cloned and sequenced to ensure it is the correct clone, the cDNA is cloned into a suitable mammalian cell expression vector. In Example 1, the mb-SCF cDNA was cloned into the pREP 8 mammalian expression plasmid from Invitrogen. It has a histidinol resistance gene as a selectable marker for the expression of the desired cloned gene. Should a second peptide wished to be expressed in the same cell line, it is advisable to use an expression vector with an alternative resistance gene as a selectable marker to ensure both plasmids are present in the same cell line. Other suitable expression vectors include: pCMV—Script from Stratgene (G418 resistance), phCMV Xi—Clone from GTS (G418 resistance), pExchanger from Stratagene (hygromycin, puromycin or neomycin resistance) and pTriEx sysem from Novagen (hygromycin).

The expression vector with the cDNA is then transformed into the viral packaging cell line and transformants selected. Should the packaging cell line already have a peptide expression vector present, then transformants are selected for both the selectable marker genes. Example 2 demonstrates how a packaging cell line is transformed and successful transformants selected.

Example 3 outlines how retroviral particles are produced and collected from viral packaging cell lines containing the mb-SCF cDNA cloned into the pREP 8 mammalian expression vector. In this way, it would be possible to produce and collect viral particles having one or more peptides incorporated into the viral envelope.

### EXAMPLE 7

### Preparation of Viral Particles Containing Antibody Fragments in the Viral Envelope

It is possible to incorporate antibody fragments into the viral particles using, for an example, the procedure outlined below.

Single Chain Fv from the Mouse Monoclonal Antibody HMFG1 and Humanised Monoclonal Antibody Hu HMFG1

The nucleotide sequences encoding the V_{H} heavy chains and VP light chains of HMFG1 and Hu HMFG1 are shown in FIG. 15 of WO 94/10323 and are given in Verhoeyen et al (1993) Immunology 78, 364-370, both incorporated herein by reference.

Nucleotide and amino acid sequences of mouse and reshaped HMFG1 variable regions. (a) Heavy chain variable region sequences for mouse and reshaped HMFG1 (Mo V_{H}-HMFG1 and Hu V_{H}-HMFG1); (b) mouse and reshaped light chain variable regions respectively (Mo V_{P}-HMFG1 and Hu V_{P}-HMFG1). Amino acids numbering and definition of the CDR and framework regions are from Kabat et al (1987) Sequences of Proteins of Immunological Interest, Edn 4, US Dept of Health and Human Services Public Health Service, NIH, Bethesda, Md. 20892, USA.

The methods described by Bird et al (1988) Science 242, 423 or Huston et al (1988) Proc. Natl. Acad. Sci. USA 85, 5879 are applied to the nucleotide sequences described in FIG. 12 to generate genes encoding ScFv for HMFG1 and ScFv for Hu HMFG1. Using the recombinant DNA techniques described above it is straightforward to fuse a region of DNA encoding the ScFv for HMFG1 and ScFv for Hu HMFG1 to the membrane spanning region of mb-SCF in clone pRep8 mb-SCF. Example 1 describes construction of pRep8 mb-SCF. The membrane spanning region of SCF is from the proteolytic cleavage site coded by exon 6 to the C-terminus of the peptide (Hamel and Westphal, 1997).

The amino acid sequences of the V_{H} and V_{L} chains of H17E2 are disclosed in AMonoclonal antibodies—applications in clinical oncology≅, pages 37-43, 1991, A. A. Epenetos, ed., Chapman & Hall, UK.

Nucleotide sequences encoding the V_{H} and V_{L} chains are readily derived from the amino acid sequence using the genetic code and an ScFv can be made from the sequences using the methods of Bird et al or Huston et al as described above.

Once the mb-SCF-ScFv clones have been constructed they are transformed into a viral packaging cell line as outlined in Example 2. Viral particles incorporating the mb-SCF-ScFv peptide can be produced as outlined in Example 3.

Viral particle which have successfully incorporated the mb-SCF-ScFv peptide can be enriched from the total population of viral particles using immunological methods, well known to those skilled in the art. For example, an antibody that recognises the ScFv molecule can be conjugated to, for example, a column or a similar supporting matrix. Total viral particles are passed through the column, and only those viral particles that have incorporated the mb-SCF-ScFv peptide will be retained. After removal of the unbound viral particles, the bound viral particles can be released from the column. Alternative methods may be used wherein the column, or a similar supporting matrix, supports an antigen which the mb-SCF-ScFv peptide can bind to. One such antigen is the polymorphic epithelial mucin (PEM) core peptide encoded by muc −1. Methods employing HMFG1 and Hu HMFG1 and/or using the epithelial mucin (PEM) core peptide are disclosed in WO 01/72336 and WO 94/10323, whose contents are herein incorporated by reference.

### EXAMPLE 8

### Use of Viral Particles of the Invention for Gene Transfer

The ability of the viral particles of the invention to facilitate transduction of quiescent target cells is tested as described below.

The TF-1 cell line was developed from an erythroleukaemic patient and probably equates to cell arrested at an early stage of megakaryocyte development (Bagnis et al., 1994). The cells can only be grown in the presence of growth factors, the most usual being IL-3 or GM-OSF to which they respond very sensitively. They also are capable of dividing in response to SCF though this response is significantly weaker. These cells can easily be rendered quiescent by withdrawal of growth factor support. We used these cells as targets for retroviral transduction, following induction of quiescence.

In this example the viral packaging cell line can produce viral particles containing nucleic acid encoding the reported gene β-galactosidase. This may be done by transfecting the same plasmid we had constructed containing the mbSCF cDNA (pRep8 mb-SCF) into retroviral producer cells transducing a retroviral vector encoding the gene for β-galactosidase (NLSlacZ). This bacterial enzyme can be used in conjunction with synthetic substrates to produce a blue staining reaction when active enzyme is present. Accordingly, in these experiments, successfully transduced cells will stain blue following virally mediated gene transfer. The resulting producer line is identified as LacJP. Immunofluorescent staining of the LacJP cell line with anti-SCF antibody will show the presence of the surface bound SCF, as discussed in Example 2.

TF-1 cells in exponential growth are removed from growth factor and incubated overnight to allow them to become quiescent. TF-1 cells are co-cultured overnight with the retroviral particles incorporating mb-SCF as discussed above. Following co-culture, the TF-1 cells are removed, collected onto microscope slides and stained for β-galactosidase production. Cells that are cocultured on the parent producer line will show no evidence for retroviral transduction. In contrast, some of the cells that are co-cultured with the SCF producer cells will be found to be positively stained. This can confirmed as cycling TF-1 cells by doubly-labelling the cells with titrated thymidine (3H). This radioactive nucleoside becomes incorporated into the DNA of dividing cells and can be detected by autoradiographic deposition of silver grains in a photographic emulsion into which the slides have been dipped. It is expected that many of the transduced TF-1 cells will also showed the presence of silver grains, indicating that cell division has taken place.

Thus, these experiments will show that expression of a surface bound growth factor by retroviral producer cell lines is able to facilitate the retroviral transduction of a quiescent target cell population and therefore enable the retroviral-mediated transfer of genes to cells that would normally be refractory to this technique.

### EXAMPLE 9

### Use of Viral Particles of the Invention for Gene Transfer In Vivo

The following example describes a procedure to use the viral particles of the invention for gene transfer. The viral particles are derived from a viral packaging cell which expresses a passenger peptide binding motif. The experiments listed below can be used to examine whether a viral particle incorporating, for example, a stem cell peptide binding motif can act as a gene transfer vector in vivo. Obviously, the tissues examined can be different depending on the cell type to which the viral particle is targeted.

Viral particles with ∃-galactosidase as a reporter gene for gene transfer, as described in Example 8, are injected in nude mice subcutaneously. Tissues are collected at 1 and 7 weeks after injection and expression of the 3-galactosidase gene assessed by a histochemical assay described below.

### Histochemical Staining for ∃-Galactosidase Activity in Cells

Cells transfected with combinations viral particles of the invention are washed with PBS and fixed in 0.1% glutaraldehyde in PBS for 10 minutes at room temperature, then washed twice in PBS, and incubated in X-gal solution (5 mM K₃Fe(CN)₆, 5 mM K₄Fe(CN)₆:3H₂0, 2 mM MgCl₂, 200 φg/ml 5-bromo-4-chloro-indolylx-∃-D-galactopyranoside in N—N-dimethylformamide) for 20-24 hours at room temperature. Expression of the ∃-galactosidase gene product in cells can be detected microscopically by an indigo blue colour.

### Staining for ∃-Galactosidase Activity in Whole Tissues

Whole tissues from mice, immediately following dissection, are washed in PBS and fixed with a solution consisting of 1% formaldehyde, 0.2% glutaraldehyde, 2 mM MgCl₂, 5 mM EGTA, 0.02% NP-40. Excess fixative is removed after 1 hour by washing in PBS, and the sample incubated overnight in the dark with staining reagents (5 mM K₃Fe(CN)₆, 5 mM K₄Fe(CN)₆:3H₂0, 2 mM MgCl₂, 0.02% NP-40, 1 mg/ml X-gal [5-bromo-4-chloro-3-∃-D-galactopyranoside; Promega, Madison Wis., USA]) in PBS. All procedures are carried out at room temperature. Samples can be examined by low power light microscopy and areas of blue stain scored in whole tissues, alternatively for confirmation, sections (1-2 mm) can be excised from areas of stained tissue and observed at high magnification.

The tissues that can be assayed for reporter gene expression include, for example, spleen, liver, kidney, brain, skin, heart, spinal chord, muscle and parotid gland.

The data will show the relative levels of blue coloration observed histochemically, and, hence, the degree of gene transfer.

Since sustained long-term expression is a desirable component of gene therapy, it is possible to attempt to assess this property in tissue culture, by monitoring ∃-galactosidasegene expression.

### EXAMPLE 10

### Gene Targeting to Cells of the Central Nervous System In Vivo Using Viral Particles of the Invention

The following example describes a procedure to use the viral particles of the invention for gene transfer to cells of the central nervous system. The viral particles are derived from a viral packaging cell which expresses a passenger peptide binding motif that can specifically bind to cells of the central nervous system. Examples of possible peptides are well known to those skilled in the art, for example, antibodies against AMPA and NMDA receptors (Pickard et al., 2000) or alpha(2A)-Adrenoreceptors (Hurt et al., 2000).

Six week old female nude (nu/nu) mice are injected subcutaneously at the back of the neck with viral particles of the invention, as described above. Animals are killed by cervical dislocation 1 or 6 weeks post injection and major organs snap frozen and stored at −70EC. One quarter of each organ is washed in phosphate buffered saline (PBS) incubated in fix (1% formaldehyde, 0.2% gluteraldehyde, 2 mM MgCl₂, 5 mM EGTA, 0.02% Nonidet P40 in PBS), washed and incubated overnight at room temperature in X-gal solution (3 mM K₄Fe(CN)₆, 3 mM K₃Fe(CN)₆, 1 mM MgCl₂, 0.05% 5-bromo-4-chlor-3-indoly-∃-D-galactopyranoside in PBS). Enzymatic activity, detected by deposition of blue product, is determined by eye or low power light microscopy.

The tissues that can be assayed for reporter gene expression include: spleen, liver, kidney, brain, skin, parotid gland. In particular, spinal tissues and other central nervous system tissues can be assayed

The data will show the relative levels of blue coloration observed histochemically, and, hence, the degree of gene transfer.

Using such a method the degree of specificity conferred by the peptide binding motif to the viral particle and the level of gene transduction can be assessed.

### EXAMPLE 11

### Other Viral Particles that can be Used in the Invention

Whilst the foregoing examples used a retroviral vector based on the PINCO and pBabe MLV retrovirus, it will be appreciated that this was only for the purposes of illustrating the invention and was not meant to limit the invention. The following are examples of other viral vectors that can be employed in the method of the invention to produce viral particles with passenger peptide binding motifs.

LentiVector is a lentivirus-based vector system produced by Oxford BioMedica. They are based on HIV and Equine Infectious Anaemia Virus (EIAV)

pVPack is a MLV-based vector system produced by Stratagene.

The Pantropic Retroviral Expression System produced by BD Biosciences uses an envelope glyocporiten derived from vesicular stomatitis virus.

Retro-X system, again produced by BD Biosciences, is derived from the MLV virus.

Other examples will be within the knowledge of skilled persons.

### EXAMPLE 12

### Administration of Viral Particles of the Invention to Mammals

The aforementioned viral particles of the invention may be administered by any conventional method including oral and parenteral (eg subcutaneous or intramuscular) injection. A preferred method of administration is by means of a nasal inhalation spray, in which the viral particles are produced as in an aerosol. Such an approach has been used in the gene therapy of cystic fibrosis as an effective way of delivering the therapeutic agent to lung epithelial cells. The treatment may consist of a single dose or a plurality of doses over a period of time.

### Pharmaceutical Formulations

Whilst it is possible for viral particles of the invention to be administered alone, it is preferable to present it as a pharmaceutical formulation, together with one or more acceptable carriers. The carrier(s) must be “acceptable” in the sense of being compatible with the viral particles of the invention and not deleterious to the recipients thereof. Typically, the carriers will be water or saline which will be sterile and pyrogen-free.

The following examples illustrate pharmaceutical formulations according to the invention in which the viral particles constitute the ‘active ingredient’.

### EXAMPLE A

### Tablet

| | | |
|---|---|---|
| Active ingredient | 100 | mg |
| Lactose | 200 | mg |
| Starch | 50 | mg |
| Polyvinylpyrrolidone | 5 | mg |
| Magnesium stearate | 4 | mg |
| | 359 | mg |

Tablets are prepared from the foregoing ingredients by wet granulation followed by compression.

### EXAMPLE B

### Ophthalmic Solution

| | | |
|---|---|---|
| Active ingredient | 0.5 | g |
| Sodium chloride, analytical grade | 0.9 | g |
| Thiomersal | 0.001 | g |
| Purified water to | 100 | ml |

| | |
|---|---|
| pH adjusted to | 7.5 |

### EXAMPLE C

### Tablet Formulations

The following formulations A and B are prepared by wet granulation of the ingredients with a solution of povidone, followed by addition of magnesium stearate and compression.

### Formulation A

| | mg/tablet | mg/tablet |
|---|---|---|
| (a) Active ingredient | 250 | 250 |
| (b) Lactose B.P. | 210 | 26 |
| (c) Povidone B.P. | 15 | 9 |
| (d) Sodium Starch Glycolate | 20 | 12 |
| (e) Magnesium Stearate | 5 | 3 |
| | 500 | 300 |

### Formulation B

| | mg/tablet | mg/tablet |
|---|---|---|
| (a) Active ingredient | 250 | 250 |
| (b) Lactose | 150 | — |
| (c) Avicel PH 101 ® | 60 | 26 |
| (d) Povidone B.P. | 15 | 9 |
| (e) Sodium Starch Glycolate | 20 | 12 |
| (f) Magnesium Stearate | 5 | 3 |
| | 500 | 300 |

### Formulation C

| | mg/tablet |
|---|---|
| Active ingredient | 100 |
| Lactose | 200 |
| Starch | 50 |
| Povidone | 5 |
| Magnesium stearate | 4 |
| | 359 |

The following formulations, D and E, are prepared by direct compression of the admixed ingredients. The lactose used in formulation E is of the direction compression type.

### Formulation D

| | mg/capsule |
|---|---|
| Active Ingredient | 250 |
| Pregelatinised Starch NF15 | 150 |
| | 400 |

### Formulation E

| | mg/capsule |
|---|---|
| Active Ingredient | 250 |
| Lactose | 150 |
| Avicel ® | 100 |
| | 500 |

### Formulation F (Controlled Release Formulation)

The formulation is prepared by wet granulation of the ingredients (below) with a solution of povidone followed by the addition of magnesium stearate and compression.

| | mg/tablet |
|---|---|
| Active Ingredient | 500 |
| Hydroxypropylmethylcellulose | 112 |
| (Methocel K4M Premium) ® | |
| Lactose B.P. | 53 |
| Povidone B.P.C. | 28 |
| Magnesium Stearate | 7 |
| | 700 |

Drug release takes place over a period of about 6-8 hours and was complete after 12 hours.

### EXAMPLE D

### Capsule Formulations

### Formulation A

A capsule formulation is prepared by admixing the ingredients of Formulation D in Example C above and filling into a two-part hard gelatin capsule. Formulation B (infra) is prepared in a similar manner.

### Formulation B

| | mg/capsule |
|---|---|
| Active ingredient | 250 |
| Lactose B.P. | 143 |
| Sodium Starch Glycolate | 25 |
| Magnesium Stearate | 2 |
| | 420 |

### Formulation C

| | mg/capsule |
|---|---|
| Active ingredient | 250 |
| Macrogol 4000 BP | 350 |
| | 600 |

Capsules are prepared by melting the Macrogol 4000 BP, dispersing the active ingredient in the melt and filling the melt into a two-part hard gelatin capsule.

### Formulation D

| | mg/capsule |
|---|---|
| Active ingredient | 250 |
| Lecithin | 100 |
| Arachis Oil | 100 |
| | 450 |

Capsules are prepared by dispersing the active ingredient in the lecithin and arachis oil and filling the dispersion into soft, elastic gelatin capsules.

### Formulation E (Controlled Release Capsule)

The following controlled release capsule formulation is prepared by extruding ingredients a, b, and c using an extruder, followed by spheronisation of the extrudate and drying. The dried pellets are then coated with release-controlling membrane (d) and filled into a two-piece, hard gelatin capsule.

| | mg/capsule |
|---|---|
| Active ingredient | 250 |
| Microcrystalline Cellulose | 125 |
| Lactose BP | 125 |
| Ethyl Cellulose | 13 |
| | 513 |

### EXAMPLE E

### Injectable Formulation

| | |
|---|---|
| Active ingredient | 0.200 g |
| Sterile, pyrogen free phosphate buffer | 10 ml |
| (pH 7.0) to | |

The active ingredient is dissolved in most of the phosphate buffer (35-40° C.), then made up to volume and filtered through a sterile micropore filter into a sterile 10 ml amber glass vial (type 1) and sealed with sterile closures and overseals.

### EXAMPLE F

### Intramuscular Injection

| | |
|---|---|
| Active ingredient | 0.20 g |
| Benzyl Alcohol | 0.10 g |
| Glucofurol 75 ® | 1.45 g |
| Water for Injection q.s. to | 3.00 ml |

The active ingredient is dissolved in the glycofurol. The benzyl alcohol is then added and dissolved, and water added to 3 ml. The mixture is then filtered through a sterile micropore filter and sealed in sterile 3 ml glass vials (type 1).

### EXAMPLE G

### Syrup Suspension

| | |
|---|---|
| Active ingredient | 0.2500 g |
| Sorbitol Solution | 1.5000 g |
| Glycerol | 2.0000 g |
| Dispersible Cellulose | 0.0750 g |
| Sodium Benzoate | 0.0050 g |
| Flavour, Peach 17.42.3169 | 0.0125 ml |
| Purified Water q.s. to | 5.0000 ml |

The sodium benzoate is dissolved in a portion of the purified water and the sorbitol solution added. The active ingredient is added and dispersed. In the glycerol is dispersed the thickener (dispersible cellulose). The two dispersions are mixed and made up to the required volume with the purified water. Further thickening is achieved as required by extra shearing of the suspension.

### EXAMPLE H

### Suppository

| | mg/suppository |
|---|---|
| Active ingredient (63 μm)* | 250 |
| Hard Fat, BP (Witepsol H15 - Dynamit Nobel) | 1770 |
| | 2020 |

*The active ingredient is used as a powder wherein at least 90% of the particles are of 63 μm diameter or less.

One fifth of the Witepsol H15 is melted in a steam-jacketed pan at 45° C. maximum. The active ingredient is sifted through a 200 μm sieve and added to the molten base with mixing, using a silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 μl stainless steel screen and, with continuous stirring, is allowed to cool to 40° C. At a temperature of 38° C. to 40° C. 2.02 g of the mixture is filled into suitable plastic moulds. The suppositories are allowed to cool to room temperature.

### EXAMPLE I

### Pessaries

| | mg/pessary |
|---|---|
| Active ingredient | 250 |
| Anhydrate Dextrose | 380 |
| Potato Starch | 363 |
| Magnesium Stearate | 7 |
| | 1000 |

The above ingredients are mixed directly and pessaries prepared by direct compression of the resulting mixture.

### EXAMPLE J

### Creams and Ointments

(see Remington: The Science and Practise of Pharmacy, 19^{th} ed., The Philadelphia College of Pharmacy and Science, ISBN 0-912734-04-3)

### EXAMPLE K

### Microsphere Formulations

The viral particles of the invention may also be delivered using microsphere formulations, such as those described in Cleland (1997), Pharm. Biotechnol. 10:1-43, Lee (2001) Curr. Opin. Biotechnol. 11:81-84, Cleland et al. (2001) J. Control. Release 72:13-24 and Takeuchi et al. (2001) Adv. Drug. Delic. Rev. 47:39-54.

### EXAMPLE L

### Dry Powder Inhalation

The viral particles of the invention may be delivered by inhalation, with the aid of a dry powder inhaler delivering micronised particles in metered quantities as described in Ansel (1999) Pharmaceutical Dosage Forms and Drug Delivery Systems, Lippincott Williams and Wilkins.

### EXAMPLE M

### Aerosol Inhalation

The viral particles of the invention may be delivered by inhalation, with the aid of a suitable inhaler delivering micronised particles in metered quantities employing a non CFC propellant as described in Ansel (1999) Pharmaceutical Dosage Forms and Drug Delivery Systems, Lippincott Williams and Wilkins.

The formulations may conveniently be presented in unit dosage form and may be prepared by any of the methods well known in the art of pharmacy. Such methods include the step of bringing into association the active ingredient (viral particles of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrate (eg sodium starch glycollate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide a desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostatis and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

### REFERENCES

Ager et al., (1996) Hum Gene Ther 7, 2157-2164
Bagnis et al., (1994) Hum Gene Ther 5, 1325-1333
Beradi et al (1995) Science 267:104-108
Bodem et al (1997) Stem Cells 15 Supp 1, 141-147
Cadd et al., (1997) Bioessays 19, 993-1000
Cosman et al., (1988) Behring Inst Mitt 149-157
Cosset and Russell (1996) Gene Therapy 3, 946-956
Cosset et al., (1995). J. Virol 69, 6314-6322
Cosset et al (1995) J Virol 69, 7430-7436.
Dimmock and Primrose, (1987) Introduction to Modern Virology Blackwell Scientific Publications, ISBN 0-632-01597-7
Etienne-Juan et al., (1992) J Gen Virol 73, 3251-3255
Garoff et al., (1998) Mirobiol Mol Biol Rev 62, 1171-1190
Goud et al., (1998) Virology 163, 251-254
Griffiths and Rottier, (1992) Semin Cell Biol 3, 367-381
Grignangi et al (1998) Cancer Res 58, 14-19
Hamel and Westphal, (1997) J Neuro-Oncology 35, 327-333
Han et al., (1995) Proc Natl Acad Sci USA 92, 9747-9751
Hatziioannou et al., (1998) J Virol 72, 5313-5317
Hatziioannou et al., (1999) Hum Gene Ther 10, 1533-1544
Hurt et al., (2000) J Biol Chem 275, 35424-35431
Kasahara et al., (1994) Science 266, 1373-1376
Kinsella and Nolan (1996) Hum Gene Ther 7, 1405-1413
Kitamura et al., (1989) Cell Physiol 140, 323-334
Lee et al., (2001) Biochem Biophys Res Commun 280, 675-683
Lee et al., (2002) Science, 297(5587):1689-92.
Lyman et al., (1995) Oncogene, 10, 149-157
Marin et al., (1996) J Virol 70, 2957-2962
Matano et al., (19950 J Gen Virol 76, 3165-3169
Miyazawa et al., (1995) Blood 85, 641-649
Morgenstern and Land, (1990) Nucleic Acid Res 18, 3587-3596
Neda et al., (1991) J Biol Chem 226, 14143-14149
Pickard et al., (2000) J Neurosci 20, 7922-7931
Roux et al., (1989) Proc Natl Acad Sci USA 86, 9079-9083
Russell et al., (1993) Nucl Acid Res 21, 1081-1085
Russell et al., (1993) Nucleic Acid Res 21, 1081-1085
Sambrook et al., MOLECULAR CLONING: A LABORATORY MANUAL. 2001. 3rd
edition Cold Spring Harbor Laboratory, Cold Spring Harbor, N.Y.
Schnierle et al., (1996) Gene Therapy 3, 334-342
Sehgal et al (1999) Gene Ther 6, 1084-1091
Seigneurin et al., (1987) Exp Hematol 15, 822-832
Sellar et al., (2003) Nat. Genet. 34(3):337-43
Somia et al., (1995) Proc Natl Acad Sci USA 92, 7570-7574
Strauss et al., (1995) Trends Microbiol 3, 346-350
Thalmeier et al., (1994) Blood 83, 1799-1807
Valsesia-Wittmann et al., (1994) J Virol 68, 4609-4619
Valsesia-Wittmann et al., (1996) J Virol 70 2059-2064

1-42. (canceled)

43. A method of making a viral particle having a modified cell binding activity comprising:
(i) providing a viral packaging cell containing viral nucleic acid encoding an enveloped viral particle, wherein said viral particle is enveloped using an envelope unable to naturally bind to cells of a species being targeted, said viral particle having a first cell binding activity wherein the viral packaging cell also contains exogenous nucleic acid encoding a passenger peptide binding moiety designed to modify said first cell binding activity of said viral particle so that the viral particle can interact with one or more different cell types than that of the unmodified viral particle; and wherein the modified cell binding activity is conferred by a peptide other than a chimaeric viral envelope polypeptide;
(ii) expressing the viral nucleic acid and exogenous nucleic acid encoding the passenger peptide binding moiety so that the passenger peptide binding moiety is provided at a cell membrane of the packaging cell and a viral particle buds from said packaging cell membrane thereby allowing the passenger peptide binding moiety to be incorporated into the viral particle to modify its first cell binding activity, wherein the passenger peptide binding moiety is selected from the group consisting of cell growth factors, antibodies or antigen-binding fragments thereof, moieties that recognize a target cell specific surface antigen, and moieties that are at least a part of a member of a binding pair comprising a target cell specific cell surface receptor and its ligand and wherein said passenger peptide is other than one naturally derived from the virus or said packaging cell.

44. A method as in claim 43 wherein the peptide binding moiety is provided at an outer plasma membrane of the cell.

45. A method as in claim 43 wherein the viral particle is derived from a retroviral vector.

46. A method as in claim 43 wherein the passenger peptide binding moiety is membrane-bound stem cell factor.

47. A method as in claim 43 wherein the viral packaging cell line comprises additional nucleic acid which can be expressed to provide a bioactive agent which is active in or on a target cell.

48. A method as in claim 47 wherein the bioactive agent has a direct or indirect cytotoxic function.

49. A method as in claim 48 wherein the bioactive agent is any one selected from the group consisting of ricin; tumour necrosis factor; interleukin-2; interferon-gamma; ribonuclease; deoxyribonuclease; Pseudomonas exotoxin A; and caspase.

50. A method as in claim 47 wherein the bioactive agent is an enzyme capable of converting a relatively non-toxic pro-drug into a cytotoxic drug.

51. A method as in claim 50 wherein the bioactive agent is either cytosine deaminase or thymidine kinase.

52. A method as in claim 43 wherein the modified cell binding activity allows the viral particle to bind to a target cell.

53. A method as in claim 52 wherein the target cell is selected from the group consisting of mammalian cells, human cells, quiescent cells, human haematopoietic stem cells, cancer cells and mammalian T-cells.

54. (canceled)
